(19) United States
(12) Patent Application Publication
(10) Pub. No.: US 2008/0021416 A1
(43) Pub. Date: Jan. 24, 2008

(54) Thin tube which can be hyperflexed by light

(75) Inventors: Tsunenori Arai, Yokohama-shi (JP); Eriko Suga, Yokohama-shi (JP); Erika Yamashita, Yokohama-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: KEIO UNIVERSITY, Tokyo (JP)

(21) Appl. No.: 11/783,185

(22) Filed: Apr. 6, 2007

Related U.S. Application Data
(63) Continuation-in-part of application No. PCT/JP05/18909, filed on Oct. 7, 2005.

(30) Foreign Application Priority Data
Oct. 7, 2004 (JP) 2004-295374

(51) Int. Cl. A61M 23/00

(52) U.S. Cl. 604/271

(57) ABSTRACT

A thin tube is provided that is inserted into a lumen of a living body so as to be used, which can detect the flexed direction of the forward end of itself with the use of a sensor disposed at such forward end upon light irradiation. The forward end of the thin tube is allowed to be flexed to a desired direction with the use of an actuator disposed at such forward end. Such thin tube for medical use is inserted into the lumen of a living body so as to be used for internal observation or internal treatment and contains a device for sensing light irradiation and/or an actuator that is operated via light irradiation at its forward end and a light transmission means, such light transmission means being used for irradiating the device and/or the actuator with light and such device or actuator functioning for monitoring and/or controlling the degree of flection of the forward end of such thin tube, is provided.

### BACKGROUND OF THE INVENTION

1. Field of the Invention

The present invention relates to a thin tube that is inserted into a lumen of a living body, such as a blood vessel, for observation and treatment of the inside of such lumen, for example. In particular, the present invention relates to a catheter that is inserted into tubular organs of a living body for use, such organs including blood vessels and digestive tracts.

2. Background Art

Hitherto, thin tubes such as catheters and the like have been used when an endoscope or the like is used for observation, diagnostics, or the like of the inside of a lumen of a living body such as a blood vessel, a digestive tract, a urinary tract, an ovarian duct, and trachea, or for therapies or the like involving internal observation and internal treatment. For instance, in order to insert a catheter into a lumen of a living body such as a blood vessel which is tortuous and branches in a complicated manner and guide the catheter to a target site, highly complex operations must be performed. Skills are necessary for handling such catheter. In order to allow a catheter to pass through a tortuous or branching site so as to guide the catheter to a target site, a method whereby a guide wire is first inserted into a catheter so that insertion of the catheter is carried out along the guide wire, a method whereby a catheter is formed into a coil and the forward end of the catheter is operated via, for example, allowing the catheter to be flexed with the use of a torque transmission tube, and other methods have recently been used. However, when trying to insert a catheter into the sigmoid colon or coronary artery, which have sites of sharp bends, it is difficult to allow the catheter to smoothly pass through such a bending site. In addition, a catheter such as a Judkins catheter, the shape of which has been adjusted to a specific bending site, has been used; however, such catheter lacks versatility. Moreover, it has been suggested that the traveling direction of the catheter be controlled by incorporating a shape-memory alloy into a catheter tube and deforming the shape-memory alloy by heat so as to allow the forward end of the catheter tip to be flexed (see Patent Documents 1 and 2) and that a balloon be applied to a catheter so that the traveling direction of the catheter is controlled by adjusting the expansion of the balloon (see Patent Documents 3 and 4).

In the cases of the above conventional catheters, the forward ends of which are operated or which are allowed to be flexed or bent under control, operability has been improved to some extent. Nonetheless, it is still necessary for an operator to handle such catheter while checking the flexed direction or the degree of flection by monitoring the forward end of the catheter. Such operation requires high-level skills. Also, a specifically designed apparatus is necessary for monitoring the degree of flection of a forward end of the catheter tip. In addition, such operation is time-consuming. In particular, the aforementioned catheters are designed in a manner such that they can be flexed in any direction. Thus, it has been difficult to control such catheters in a specific direction since they can be flexed in any direction. Further, in cases of catheters comprising a shape-memory alloy, an electric current is allowed to flow into a shape-memory alloy section for heat generation. In such cases, strict insulating is necessary to prevent leakage of electric current into the heart.

In addition, catheters comprising a double lumen tube have been used. However, such catheters comprising a double lumen tube have been exclusively balloon catheters (see Patent Document 5).

Patent Document 1 JP Patent Publication (Kokai) No. 61-255669 A (1986)

Patent Document 2 JP Patent Publication (Kokai) No. 7-323091 A (1995)

Patent Document 3 JP Patent Publication (Kokai) No. 8-47539 A (1996)

Patent Document 4 JP Patent Publication (Kokai) No. 2003-230629 A

Patent Document 5 JP Patent Publication (Kokai) No. 09-028808 A (1997)

### SUMMARY OF THE INVENTION

It is an objective of the present invention to provide a thin tube that is inserted into a lumen of a living body so as to be used, such tube being able to detect the flexed direction of its forward end with the use of a sensor disposed at such forward end upon light irradiation. The forward end of such thin tube is allowed to be flexed in a desired direction with the use of an actuator disposed at such forward end. It is another objective of the present invention to provide a thin tube that can detect the traveling direction and is actively flexed to a direction in which the forward end of the thin tube has been passively flexed when coming into contact with a lumen.

As described above, in the cases of the above conventional catheters, the forward end of which can be controlled, a complicated mechanism is necessary. In addition, operation of such forward end requires skills and is time-consuming. Further, in a case of a catheter in which a shape-memory alloy is used such that it becomes possible to operate the forward end, it is necessary to allow an electric current to flow into the shape-memory alloy. Thus, strict insulating is necessary for preventing leakage of electric current.

The inventors of the present invention have made intensive studies of a thin tube such as a catheter with a forward end that can readily and rapidly be operated. When the flexed forward end of a catheter is irradiated with light, the inner wall (opposite to the side to which the forward end is flexed) of the flexed forward end of the catheter is exposed to light. The inventors of the present invention have found that the flexed direction of the forward end of a catheter can be detected in such case by measuring light or temperature increases at a site exposed to light upon light irradiation. Further, the present inventors have found that it is possible to deform the shape of the forward end of a catheter and control the flection of the forward end of the catheter so as to control the traveling direction of the catheter with the use of a material that can be deformed by light and heat generated upon light irradiation. For instance, when a catheter is inserted into a lumen, the forward end of the catheter comes into contact with the bending section of the lumen so as to become lightly flexed. In such case, a means of irradiating the inside of a lumen of a catheter with light, such as a laser or the like, is provided in a manner such that light irradiation takes place in the traveling direction of the catheter. Thus, light irradiation is performed when the forward end of the catheter is lightly flexed so that a part of the inner wall of the catheter (such part being located opposite to the side to which the forward end of the catheter has been flexed) is always irradiated with light. Thus, a material (light-absorbing material) that generates heat by absorbing light and a material (deformable material) the shape or the volume of which varies depending on heat are disposed at a position subjected to light irradiation in a manner such that they are allowed to come into contact with each other for heat conduction. Accordingly, heat generated upon light irradiation changes the shape of such deformable material, resulting in change in flection of the forward end of the catheter. Therefore, it becomes possible to regulate the traveling direction of the catheter.

The inventors of the present invention have designed a catheter in the following manner: a light-absorbing material and a deformable material are allowed to come into contact with each other over the whole circumference of the forward end of the catheter; a light-absorbing material, which is located opposite to the side to which the forward end of the catheter has been flexed, is irradiated with light when the forward end is lightly flexed after coming into contact with a lumen, for example; and generated heat is conducted to the deformable material. Further, they have designed a catheter having a double lumen structure in a manner such that a light-absorbing material and a deformable material are allowed to come into contact with each other at one side of the forward end of an inner catheter, and the forward end of the inner catheter is moved to the inside of an outer catheter such that the light-absorbing material and the deformable material are irradiated with light, so that the forward end of the catheter is allowed to be flexed in a desired direction. The inventors of the present invention have found that, in such case, the forward end of the catheter is allowed to be further flexed (hyperflexed) to the side to which the forward end has been lightly flexed in such case. This is because, when such deformable material is applied to a catheter, the deformable material extends in the traveling direction (longitudinal direction) of the catheter so that the part of the catheter to which the deformable material has been applied is allowed to be flexed as the

[7] The thin tube for medical use according to [6], wherein the deformable material absorbs light so as to generate heat so that the deformable material can be deformed by heat.

[8] The thin tube for medical use according to [6], containing a light-absorbing material that absorbs light so as to generate heat and a deformable material that can be deformed by heat in a manner such that the light-absorbing material and the deformable material are allowed to come into contact with each other for thermal conduction at the forward end of the thin tube and a light transmission means; wherein

the forward end of the thin tube can be flexed by irradiating the light-absorbing material with light from the light transmission means so as to cause deformation of the deformable material due to conduction of heat that is generated from the light-absorbing material.

[9] The thin tube for medical use according to [6], comprising a deformable material disposed in a continuous manner or at certain intervals over the whole circumference of the forward end of the thin tube.

[10] The thin tube for medical use according to [6], wherein the deformable material that can be deformed is a bimetal or shape-memory alloy.

[11] The thin tube for medical use according to [6], wherein the deformable material that can be deformed is a polymer gel actuator.

[12] The thin tube for medical use according to [6], wherein the flexed angle of the forward end of the thin tube can be controlled by changing the intensity of irradiated light so as to change the strength of the deformable material to be deformed.

[13] A double lumen thin tube for medical use, which is inserted into a lumen of a living body so as to be used for observation or treatment, comprising an inner thin tube and an outer thin tube, wherein the inner thin tube is the thin tube for medical use according to [1].

[14] The double lumen thin tube for medical use according to [13], which is inserted into a lumen of a tubular object or a space of a construct so as to be used, wherein:

the inner thin tube comprises a deformable material that can be deformed by light irradiation so as to serve as an actuator that is operated via irradiation of light from the forward end, and the deformable material is deformed by the action of light irradiated from a light transmission means in the thin tube, so that the forward end of the thin tube can be flexed;

the actuator that is operated via irradiation of light from the inner thin tube is provided at only one side of the inner thin tube;

the inner thin tube is provided in an outer thin tube in a movable manner in the anteroposterior direction and in a rotatable manner; and

the actuator of the inner thin tube is disposed on the same side as or opposite to the side to which the inner thin tube is allowed to be flexed by allowing the inner thin tube to be moved in the anteroposterior direction or rotated in the outer thin tube, and light irradiation is performed, so that the inner thin tube is allowed to be flexed.

[15] The double lumen thin tube for medical use according to [13], wherein the inner thin tube is a torque transmission tube.

[16] The double lumen thin tube for medical use described above, wherein:

the device for sensing light irradiation that is disposed in the forward end of the outer thin tube is a light sensor for sensing light irradiation or a thermal sensor that is continuously or intermittently (e.g., at certain intervals) provided over the whole circumference of the forward end of the thin tube;

the thin tube can detect that the forward end of the thin tube has been flexed to the side opposite to the side subjected to light irradiation by monitoring light irradiated from a light transmission means contained in the thin tube with the light sensor or monitoring temperature increase due to light irradiation with the thermal sensor and monitoring a part of the whole circumference of the forward end of the thin tube subjected to light irradiation;

the inner thin tube comprises a deformable material that can be deformed by light irradiation so as to serve as an actuator that is operated via irradiation of light from the forward end, and the deformable material is deformed by the action of light irradiated from a light transmission means in the thin tube, so that the forward end of the thin tube can be flexed;

the inner thin tube is provided in an outer thin tube in a movable manner in the anteroposterior direction and in a rotatable manner; and

the actuator of the inner thin tube is disposed on the side opposite to the flexed direction of the forward end of the thin tube that has been monitored with the use of the outer thin tube by allowing the inner thin tube to be moved in the anteroposterior direction or rotated in the outer thin tube, and light irradiation is performed, so that the inner thin tube is allowed to be further flexed.

[17] The double lumen thin tube for medical use according to [16], wherein the inner thin tube is a torque transmission tube.

[18] A method for inserting the thin tube for medical use according to any one of [1] to [13] into a lumen of a living body, comprising the steps of:

(a) inserting the thin tube into a lumen of a living body;

(b) irradiating a device and/or actuator of the forward end of the thin tube with light by a light transmission means that is disposed in the thin tube so as to allow the forward end of the thin tube to be flexed when the thin tube comes into contact with the inner wall of the lumen of a living body so that it becomes difficult to insert the forward end of the thin tube; and

(c) further inserting the thin tube into the lumen of a living body.

This description includes part or all of the contents as disclosed in the description of Japanese Patent Application No. 2004-295374, which is a priority document of the present application.

### BRIEF DESCRIPTION OF THE DRAWINGS

FIG. 1A shows a condition in which the thin tube of the present invention has been inserted into a blood vessel.

FIG. 1B shows a condition in which the forward end the thin tube of the present invention is allowed to be flexed by light irradiation.

FIG. 2A shows a condition in which the double lumen thin tube of the present invention has been inserted into a blood vessel.

FIG. 2B shows a condition in which the forward end of the double lumen thin tube of the present invention is allowed to be flexed by light irradiation.

FIG. 3A illustrates a method for introducing a thin tube into a branching site of a blood vessel with the use of a guide wire.

FIG. 3B illustrates a method for introducing the double lumen thin tube of the present invention into a branching site of a blood vessel.

FIG. 3C illustrates a method for introducing the double lumen thin tube of the present invention combined with a guide wire into a branching site of a blood vessel.

FIG. 4 shows a picture of a thin tube before laser irradiation, such thin tube being subjected to an experiment of flection of a thin tube by irradiation of a laser from the inside of the thin tube.

FIG. 5 shows a picture of a thin tube after laser irradiation, such thin tube being subjected to an experiment of flection of a thin tube by irradiation of a laser from the inside of the thin tube.

FIG. 6 shows a picture of a thin tube before laser irradiation, such thin tube being subjected to an experiment of flection of a thin tube by irradiation of a laser from the outside of the thin tube.

FIG. 7 shows a picture of a thin tube after laser irradiation, such thin tube being subjected to an experiment of flection of a thin tube by irradiation of a laser from the outside of the thin tube.

FIG. 8 shows a picture of a thin tube before laser irradiation, such thin tube being subjected to an experiment of flection of a thin tube in a simulated blood vessel.

FIG. 9 shows a picture of a thin tube after laser irradiation, such thin tube being subjected to an experiment of flection of a thin tube in a simulated blood vessel.

FIG. 10 shows results of the measurement of the temperature of a tube subjected to laser irradiation.

FIG. 11 shows results of the measurement of the temperature of a tube subjected to laser irradiation.

FIG. 12 shows results of the observation of a blood vessel with the use of the thin tube of the present invention into which an angioscope has been incorporated.

FIG. 13 shows an endoscope apparatus, such endoscope irradiating the inside of a lumen with high-intensity pulsed light and generating vapor bubbles so as to be able to temporarily exclude liquid in the lumen.

FIG. 14 shows a cross section of a catheter of an endoscope apparatus, such endoscope irradiating the inside of a lumen with high-intensity pulsed light and generating vapor bubbles so as to be able to temporarily exclude liquid in the lumen.

FIG. 15 shows the apparatus used in Examples 4 to 6 described below.

FIG. 16 shows vapor bubbles induced by a laser.

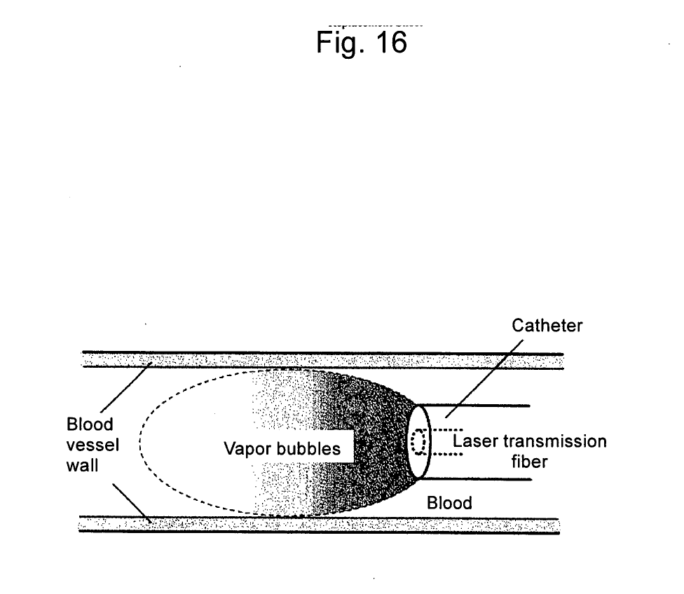

FIG. 17 shows the temporal relationship among high-intensity pulsed light irradiation, generation of vapor bubbles, and illumination light flash.

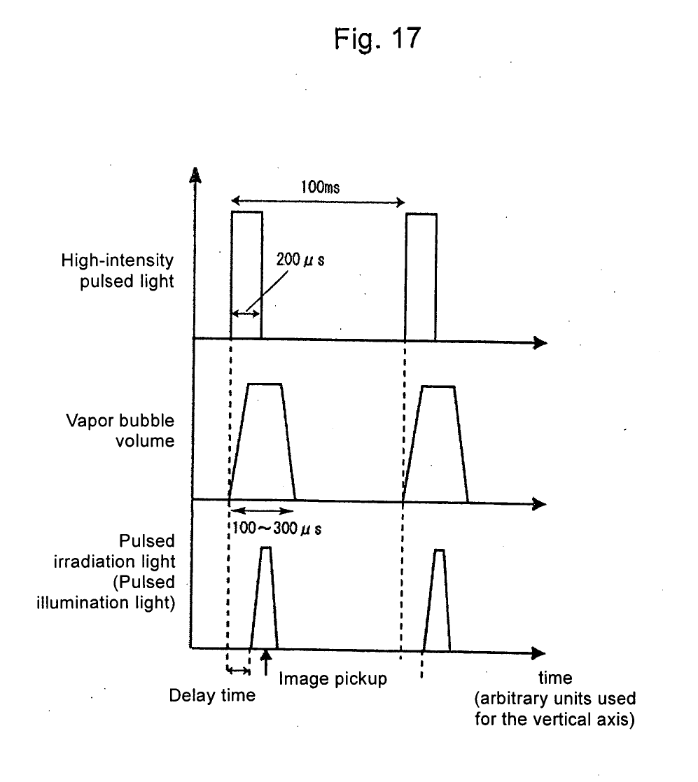

FIG. 18 shows a picture of the observation of the inside of a silicone tube filled with milk at delay time of 70 μs.

FIG. 19 shows a picture of the observation of the inside of a silicone tube filled with milk at delay time of 140 μs.

FIG. 20 shows the relationship among delay time between laser irradiation and pulsed illumination, the size of an image of image pickup, and the relative degree of lightness upon image pickup of the inside of a silicone tube filled with milk following laser irradiation.

### DESCRIPTION OF SYMBOLS

1 THIN TUBE
2 LIGHT-TRANSMITTING FIBER
3 BLOOD VESSEL
4 LIGHT-ABSORBING/DEFORMABLE MATERIAL
5 LIGHT
6 OUTER THIN TUBE
7 INNER THIN TUBE
8 GUIDE WIRE
9 CATHETER
10 HIGH-INTENSITY PULSED LIGHT-TRANSMITTING FIBER
11 TRANSMITTING FIBER FOR LIGHT FOR OBSERVATION
12 HIGH-INTENSITY PULSED LIGHT IRRADIATING PART
13 ILLUMINATING PART FOR LIGHT FOR OBSERVATION
14 HIGH-INTENSITY PULSED LIGHT SOURCE
15 SOURCE OF LIGHT FOR OBSERVATION
16 DELAY PULSE GENERATOR
17 ILLUMINATING PART
18 LIGHT GUIDE (FOR ILLUMINATION)
19 PULSE ILLUMINATING LIGHT SOURCE
20 OBSERVATION PART
21 IMAGE GUIDE
22 IMAGING DEVICE
23 PROCESSING PART
24 MONITOR
25 LUMEN (FOR INJECTING SALINE)
26 CATHETER SHEATH
27 LASER TRANSMISSION FIBER
28 IMAGE GUIDE
29 LIGHT GUIDE
30 SMALL-DIAMETER ENDOSCOPE
31 SHEATH
32 Ho:YAG LASER GENERATOR
33 FLASH LAMP
34 CONDENSING LENS
35 DELAY GENERATOR
36 CCD CAMERA
37 MONITOR
38 PIG CORONARY ARTERY

### DESCRIPTION OF THE PREFERRED EMBODIMENTS

The present invention relates to a thin tube that is inserted into a lumen of a living body for the purposes of, for example, observing and treating a target site, such thin tube being capable of detecting the flexed direction of the forward end thereof by light irradiation. Further, the thin tube of the present invention can be operated in a manner such that the forward end thereof can be freely flexed. Thus, in a case of a flexed lumen, the forward end of the thin tube of the present invention is allowed to be readily flexed in a desired direction through by performing light irradiation alone at a certain intensity for a certain period of time from the tip of a light transmission fiber accommodated in the thin tube without complicated operations. Even in a case of a lumen that is tortuous and/or branches in a complicated manner, it is possible to smoothly insert the thin tube into the lumen in the traveling direction of the lumen so as to guide the thin tube to a target site. Further, when the forward end of the thin tube is passively flexed at a bending site of a lumen, it is possible to allow the forward end of the thin tube to be further actively flexed in the flexed direction by light irradiation at a certain intensity for a certain period of time from the tip of a light transmission fiber accommodated in the thin tube. As a result, it is possible to change the traveling direction of a catheter at, for example, a branching site, site of stenosis, or aneurysm neck of a blood vessel. That is, the thin tube of the present invention has a flection mechanism. In addition, since it is possible to allow the thin tube of the present invention to be further flexed in the direction in which the tube has been already flexed, the thin tube of the present invention can be referred to as a thin tube that is hyperflexed by light.

Examples of the thin tube of the present invention include medical catheters and medical endoscopes. Examples of medical catheters include any types of medical catheters such as heart catheters, blood vessel catheters, kidney catheters, intravenous catheters, and neurocatheters. Lumens into which such medical catheters can be inserted are lumens of a living body. Depending on purpose, medical catheters are applied to blood vessels, urinary tracts, digestive tracts, trachea, ovarian ducts, and the like. Also, examples of medical endoscopes include cardioscopes, angioscopes, large intestine endoscopes (colonoscopes), upper gastrointestinal endoscopes, tubal endoscopes, and neuroendoscopes. In general, an endoscope is used combined with a catheter-like tube. Thus, the term “catheter” includes any endoscope. The aforementioned medical catheters and medical endoscopes may comprise a variety of treatment apparatuses such as balloons.

The size of the above thin tube is not limited, and thus an adequate size can be selected depending on the type and the size of the lumen into which the tube is inserted. Also, the material used for such thin tube is not limited, and thus synthetic resins, metals, and any combination thereof can be adequately used as long as such material has flexibility to such an extent that the tube can be flexed in a bending lumen according to the degree of bending of the lumen. Examples of such material include polyethylene, polyethyleneterephthalate, polypropylene, polyvinyl chloride (PVC), polyurethane, polyamide, polyamide elastomer, polyimide, polyimide elastomer, fluororesin, silicone, and natural rubber. In addition, in cases of metals, mesh-like or coiled metals are used. Such metals may be combined with the above resins.

It is possible to produce the thin tube of the present invention by processing the forward end of a thin tube that is a catheter or the like that has been conventionally used for the aforementioned purposes.

Examples of such thin tube include blood vessel catheters. Such catheters that can be used have sizes of 3 Fr. to 6 Fr. and lengths of approximately 1 to 2 m.

The thin tube of the present invention has at least one lumen through the entire length thereof or through almost the entire length thereof. The thin tube of the present invention is provided with a deformable material that can be deformed by heating the forward end of the thin tube and a light-absorbing material that can generate heat by absorbing light. Thus, it is possible to allow the thin tube of the present invention to be flexed and reach a target site in a lumen that is tortuous and branches in a complicated manner or a target site inside machinery or a construct having a complicated inner structure by irradiating the light-absorbing material with light such as laser light and conducting heat generated by the light-absorbing material to the deformable material so as to deform the deformable material. Herein, the forward end of a thin tube is referred to as the distal end of a thin tube in some cases, indicating the proximal part of the farthest forward end of a thin tube (such part being a distance of approximately several tens of centimeters from the farthest forward end). In addition, the end part opposite to the above forward end (such part being a distance of approximately several tens of centimeters from the farthest forward end) is referred to as a handgrip part or proximal end part. The aforementioned thin tube is connected to an operation part of the handgrip part, which is used for operating the movement of the thin tube. The lumen inside the thin tube accommodates an optical fiber used for light irradiation in order to heat a deformable material, an optical fiber that functions as an endoscope, and a variety of apparatuses, including a drug administration apparatus, such apparatuses being used for treating a lumen of a living body and repairing a lumen of a pipe or the like and the inside of machinery or a construct.

Further, the thin tube of the present invention may have a double lumen thin tube structure comprising an inner thin tube and an outer thin tube. In such case, it is possible to move an inner thin tube inside an outer thin tube in the anteroposterior direction. Furthermore, it is possible to rotate such inner thin tube in such outer thin tube. Such rotational movement can be realized upon hand operation with the use of a torque transmission tube as the inner thin tube. In addition, regarding the rotational movement of the thin tube, rotation of the entire thin tube does not actually take place in a uniform manner; however, rotation of the forward end of the thin tube takes place due to torsion of the thin tube. According to the present invention, the rotational movement of the forward end of the thin tube due to torsion of the thin tube is described as “rotational movement” or “movement in the rotational direction” of the thin tube. Also, in the case of a double lumen thin tube structure, it is possible to determine the adequate sizes of an outer thin tube and an inner thin tube.

Examples of a “device for sensing light irradiation” that is disposed in the forward end of the thin tube of the present invention include a light sensor and a thermal sensor. A light sensor directly detects irradiated light. The type of such light sensor is not limited. Any light sensor can be used as long as it can detect light. Examples thereof include: photoconductive elements such as CdS; photovoltaic elements such as phototransistors, photodiodes, and photothyristors; light-receiving elements such as photovoltaic pickup tubes and photomultiplier tubes; and photocouplers such as photodiode arrays, PSD, CCD image sensors, MOS image sensors, and DJPD. Such thermal sensor detects temperature increases at a part subjected to light irradiation. Therefore, it is necessary for a thermal sensor to absorb light so as to generate heat by itself or to be provided with a material that absorbs light so as to generate heat in a manner such that the material comes into contact with the thermal sensor at a site irradiated with light. According to the present invention, the term “thermal sensor” involves a material that absorbs light so as to generate heat. Such light-absorbing material will be described below. Examples of such thermal sensor that can be used include, but are not limited to, a thermocouple, a thermo-sensitive semiconductor, and an infrared ray-sensitive sensor. Such device for sensing light irradiation is continuously or intermittently (e.g., at certain intervals) provided to the whole circumference of the forward end of a thin tube. In addition, an anteroposterior position in the axial direction on the thin tube subjected to light irradiation (anteroposterior direction or longitudinal direction) varies depending on the degree of flection. Thus, a plurality of such devices may be provided in the anteroposterior direction. With the application of such device, a part subjected to light irradiation can be detected by light or temperature. In a case in which the forward end of the thin tube is not flexed, light travels in a straight direction so that the above device is not irradiated with light, resulting in no detection of light and heat. Meanwhile, in the case in which the forward end of the thin tube is flexed, the inner wall of a flexed part of the thin tube is irradiated with light such that the aforementioned device provided at the flection of the thin tube can detect the part irradiated with light. Accordingly, it is possible to detect that the forward end of the thin tube is flexed to the direction opposite to the side on which the part irradiated with light is located. Further, a thin tube introduced into a lumen is flexed, mostly in cases in which the forward end of the thin tube comes into contact with the lumen. In such cases, it is possible to detect with which side of a lumen a thin tube comes into contact with the use of a light sensor or thermal sensor. For instance, when a thermal sensor is provided over the whole circumference of the forward end of a thin tube, the maximum temperature is detected by a thermal sensor subjected to light irradiation such that thermal sensors in the vicinity of the thermal sensor detect temperatures lower than the maximum temperature. Signals sensed by a device for sensing light irradiation can be detected with the use of a lead wire provided in the thin tube in a manner such that the lead wire electrically communicates between the above device and a detection apparatus on a handgrip side.

When a groove or paint is provided on a thin tube in the longitudinal (axial) direction for marking, it is possible to detect at a handgrip part with which side of a lumen the thin tube has come into contact (i.e., the side opposite to the side to which the thin tube has been flexed). Thus, since it is possible to detect at a handgrip part with which side of a lumen a thin tube has come into contact (i.e., the side opposite to the side to which the thin tube has been flexed), it is possible to control the position of an actuator when introducing a thin tube having a forward end comprising an actuator that is operated via light irradiation as described below. In addition, the aforementioned device for sensing light irradiation may be contained in a conventional a thin tube (catheter) that can be flexed only in one direction. In such case, it is possible to know the positional relationship of the side on which a thin tube has come into contact with a lumen and the side to which a thin tube can be flexed due to the above marking. Thus, it is possible to allow a thin tube to travel in the bending direction of a bending site of a lumen by moving the thin tube in a manner such that the side to which a thin tube can be flexed is located opposite to the side on which a thin tube has come into contact with a lumen.

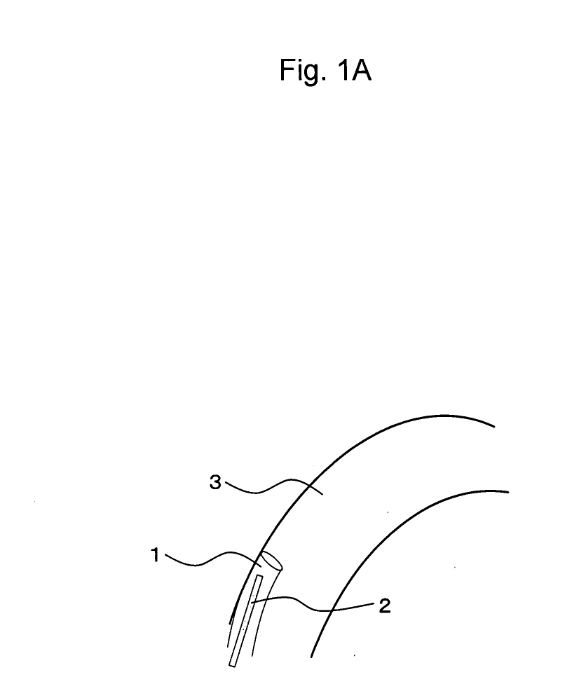

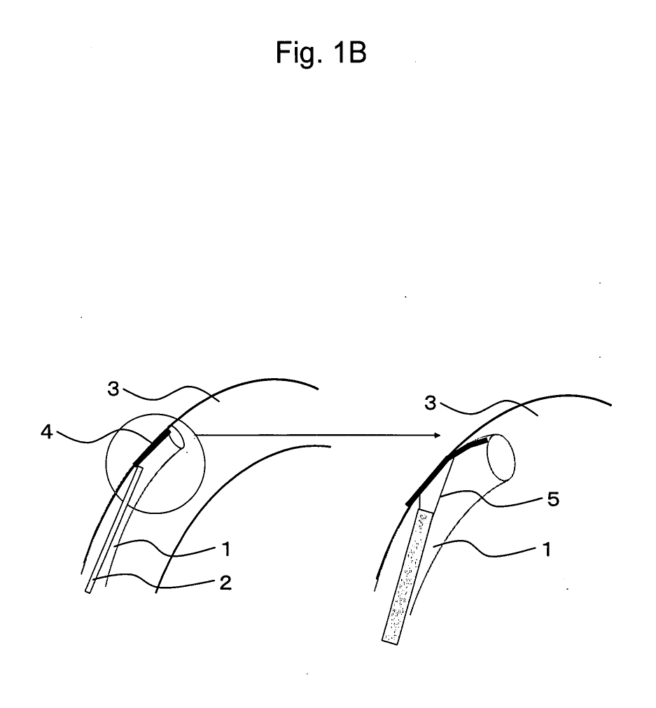

The term “actuator” indicates an element or apparatus that converts a certain type of operation energy into a mechanical quantity according to input signals. The term “actuator that is operated via light irradiation” of the apparatus of the present invention indicates a device that can be operated in a manner such that the forward end of a thin tube is allowed to be flexed due to light irradiation. Examples thereof include deformable materials. Deformable materials can be deformed or volumes thereof can be changed by heat. According to the present invention, deformable materials may exist at least on the side opposite to the side on which the forward end of a thin tube is flexed. A deformable material is extended by heat so that the forward end of a thin tube is partially extended. Accordingly, the forward end of the thin tube is flexed to the side opposite to the side on which the extended deformable material is located. In some cases, the deformable material of the present invention is referred to as extensible material. Such deformable material enables the forward end of a thin tube to be flexed due to the deformation thereof. Thus, it is necessary that the strength of flection of such deformable material be equivalent to or exceed the rigidity of a thin tube such that the thin tube is allowed to be flexed. In a typical example, a resin-made thin tube and a metal-made deformable material are used in combination. Further, since a deformable material is extended as a result of its deformation, the forward end of a thin tube is allowed to be flexed. Thus, a deformable material is provided in a manner such that it can be extended in the longitudinal direction of a thin tube (in the direction in which the thin tube travels when inserted). For instance, a deformable material that has been processed into a string or reed form may be provided to the inner or outer wall of a thin tube (see FIG. 1B). In addition, such deformable material may be provided to a part of the whole circumference of the wall of a thin tube or may be provided intermittently (e.g., at certain intervals) or continuously to the whole circumference of the wall of a thin tube. In a case in which the material is provided to the whole circumference of a thin tube as descried above, it is possible to allow the thin tube to be flexed to the side opposite to the side subjected to light irradiation when an arbitrary side of the inner wall of a thin tube that is subjected to light irradiation. Further, a plurality of deformable materials may be provided to a thin tube in the anteroposterior (longitudinal) direction. In such case, it is possible to allow a thin tube to be flexed at a desirable site thereof in the anteroposterior (longitudinal) direction with the use of deformable materials to be irradiated with light.

Examples of such deformable materials include bimetals and shape-memory alloys. A bimetal is made in a manner such that at least two types of metal plates having different thermal expansion rates are attached together. Such bimetal is deformed upon temperature change in a manner such that the bimetal is bent to the side of a metal plate having a smaller thermal expansion rate. In addition, a combination of 3 types of metals is referred to as a trimetal. However, according to the present invention, the term “bimetal” includes all combinations of 2 or more types of metals. When a bimetal is provided at the forward end of a thin tube in a manner such that the metal of the bimetal having the smallest thermal expansion rate is located on the interior side of a flexed part, the bimetal bends to the side of such metal having the smallest thermal expansion rate so that the forward end of a thin tube is flexed to the same side. In cases of bimetals, the curvature coefficient and the temperature range for use are determined depending on the combination of metals. Thus, the necessary curvature coefficient and temperature range are determined according to the application of a thin tube. Based on such coefficient and temperature, a bimetal to be used can be selected. Some lumens have sharp bending sites. Therefore, the maximum extent of flection of a thin tube is preferably large. In this regard, a bimetal having a large curvature coefficient is preferable. The larger the curvature coefficient, the higher the degree of curvature (displacement) of a bimetal. Herein, the term “the degree of curvature” indicates a distance between the position of the most bent part of the forward end of a bimetal and the original horizontal position of such part when a straight and horizontal bimetal is heated so as to be bent. In addition, the degree of curvature varies depending on temperature. The higher the temperature, the larger the degree of curvature. Thus, by controlling temperature increase, the aforementioned displacement can be freely changed. That is, the degree of flection of the forward end of a thin tube can be controlled. For instance, a curvature coefficient of the bimetal used for the thin tube of the present invention is 5×106/K or more and preferably 10×106/K or more from room temperature to 100° C. Such temperature range for use varies depending on the application of the thin tube. For instance, when the thin tube is a thin tube such as a medical catheter that is inserted into a lumen of a living body, the thin tube is preferably used at approximately 60° C. or less. Examples of the bimetal used for the thin tube of the present invention include BR-1 (NEOMEX) or the like.

In addition, the aforementioned shape-memory alloys are metals that can be deformed at a certain temperature upon heating. Any conventional shape-memory alloy can be used. For instance, NI-TI (nickel-titanium) and CU-ZN-AL (copper-zinc-aluminium) shape-memory alloys are available. Such a shape-memory alloy is provided in a manner such that the shape-memory alloy is allowed to be extended in the longitudinal direction of a thin tube as described above. In order to realize such configuration, it is convenient for a shape-memory alloy that is in a string or reed form at a high temperature to be formed into a coil or partially bent at a low temperature such that it has a length that is shorter than its original length. In such case, a shape-memory alloy that is in a coil form or is partially bent is extended upon heating so that the forward end of a thin tube can be flexed. The temperature for deforming a shape-memory alloy can be adequately determined according to the application of a thin tube. For instance, in a case of a thin tube that is inserted into a lumen of a living body, the desirable temperature is approximately 60° C. or less.

Other examples of the above deformable materials that can be used include not only the aforementioned bimetals or shape-memory alloys made of metals but also polymer materials. As an example of a polymer-made deformable material, a polymer gel actuator made of a polymer gel material can be used. Such actuator experiences changes in volume, extension/contraction, and/or bending due to environmental changes in temperature, light, and the like. Examples of such polymer gel actuator include: azobenzene-polyacrylic acid ethyl gum (which contracts in the presence of ultraviolet light and extends in the presence of visible light) that experiences changes in volume, extension/contraction, and/or bending upon light irradiation; butyl methacrylate-acrylamide-acrylic acid monomer (which contracts at low temperatures and expands at high temperatures) that experiences changes in volume, extension/contraction, and/or bending due to temperature changes; and γ-ray crosslinked PVME (which contracts at low temperatures and expands at high temperatures).

These polymer gel actuators may be formed by processing so as to be provided to the forward end of a thin tube in a manner such that the forward end of a thin tube is flexed due to changes in volume, extension/contraction, and/or bending of a polymer actuator. In order to cause a polymer actuator to experience changes in volume, extension/contraction, and/or bending, light irradiation may be performed in the case of an actuator that experiences changes in volume, extension/contraction, and/or bending due to light. In addition, in the case of an actuator that experiences changes in volume, extension/contraction, and/or bending due to temperature changes, a light-absorbing material in contact with the actuator is irradiated with light such that the light-absorbing material is allowed to generate heat and the generated heat is conducted to the actuator. Alternatively, such actuator is irradiated with light such that the actuator is allowed to generate heat by itself. Examples of such polymer gel actuator that can be used are described in Tadokoro, Journal of the Robotics Society of Japan, Vol. 15, No. 3, pp. 318-322, 1997.

The above light-absorbing material that absorbs light so as to generate heat is not limited. However, the material used is determined based on a combination of such material and the wavelength of light to be used.

The above light-absorbing material absorbs light so as to cause thermal conduction to the aforementioned deformable material. In order to cause efficient thermal conduction, a light-absorbing material having a high degree of thermal conductivity is preferable. For the purpose of allowing such light-absorbing material to cause thermal conduction to the aforementioned deformable material, it is necessary for both materials to come into contact with each other. Such contact may be partial contact. However, preferably, the materials are in contact with each other while sharing a large contact surface for efficient thermal conduction. For instance, the light-absorbing material and the deformable material may be processed into pieces of almost the same size so that they can be attached together for use. The light-absorbing material is disposed inside the deformable material so as to come into contact therewith. This is because the light-absorbing material receives light from a light transmission fiber that is disposed in the lumen inside a thin tube. In addition, the light-absorbing material may exist inside a thin tube. In such case, the light-absorbing material is preferably disposed on the wall surface of a thin tube in a manner such that at least a part of the light-absorbing material is subjected to direct light irradiation. Further, the deformable material may be covered with the light-absorbing material. In such case, the entirety of the deformable material may be covered. Alternatively, only a part of the deformable material subjected to light irradiation may be covered. Furthermore, even in a case in which the light-absorbing material and the deformable material do not directly come into contact with each other, it can be said that they are in contact with each other if heat generated in the light-absorbing material can be conducted to the deformable material.

Moreover, in the apparatus of the present invention, the light-absorbing material and the deformable material may be the same material. Examples of such material that can be used as a deformable material include not only bimetals and shape-memory alloys made of metal but also polymer materials. Such metals and polymer materials can absorb light so as to generate heat. Thus, the deformable material per se can be used as the light-absorbing material.

According to the present invention, a deformable material that serves as a light-absorbing material is referred to as a light-absorbing/deformable material (light-absorbing/extensible material) in some cases. Also, a deformable material in contact with a light-absorbing material is referred to as light-absorbing/deformable material (light-absorbing/extensible material) in some cases.

In addition, the forward end of a thin tube may be produced using a light-absorbing material. In such case, a deformable material is provided at the forward end of a thin tube so that such deformable material comes into contact with a light-absorbing material, resulting in heat conduction from the light-absorbing material to the deformable material.

In the apparatus of the present invention, the type of light beam that can be detected by a light sensor or thermal sensor, and the type of light beam used for light irradiation for causing a light-absorbing material to generate heat are not limited. However, a continuous or pulsed laser light beam or a light beam that is generated by a wavelength-variable optical parametric oscillator (OPO) is preferable. Preferably, for instance, frequency-doubled laser waves are used. Examples of lasers include a semiconductor laser, a dye laser, and a wavelength-variable near-infrared laser. The above light beam may be a pulsed light beam of a pulsed laser or the like or a continuous light beam of a continuous laser or the like. In addition, irradiation with continuous light may be intermittently performed using a light chopper such that a pulsed light beam is provided. In the apparatus of the present invention, a continuous light beam of a semiconductor laser is preferably used.

A means of transmitting light into a lumen comprises a light irradiation means that is provided in the vicinity of the forward end of a thin tube and an optical fiber (e.g., a quartz fiber, a plastic fiber, or a hollow pathway used for light transmission) whereby light is transmitted from a light generating device to the light irradiation means. According to the present invention, a quartz fiber is desirably used.

A quartz fiber is contained in a lumen of a thin tube. One end of the fiber is connected to a light generating device. The other end of the fiber is connected to a light irradiation means. The fiber used in the present invention is adequately selected depending on the application and the diameter of the relevant thin tube. Fibers having widely different diameters such as an extremely thin fiber having a diameter of approximately 0.05 to 0.3 mm and a fiber having a visible diameter can be used as long as they can be accommodated in a thin tube so as to be used for transmission of light energy. In addition, a fiber used for light irradiation to a device for sensing light irradiation may differ from a fiber used for light irradiation to an actuator that is operated via light irradiation. In such case, the former fiber should be thicker than the latter fiber. With the use of such thicker fiber, flection of the forward end of a thin tube is monitored and the thicker fiber is temporarily removed. Next, a thinner fiber used for light irradiation to an actuator that is operated via light irradiation may be inserted.

The direction of light irradiation may be parallel to the longitudinal direction of a thin tube. In addition, the direction of light irradiation from the light irradiation means may be flexible and controllable. In the former case, light irradiation is performed when the forward end of a thin tube is lightly flexed so that the thin tube is allowed to be further flexed in a direction identical to the direction in which the forward end of a thin tube has been lightly flexed. In the latter case, the direction of light irradiation is changed such that the forward end of a thin tube is allowed to be flexed in a desired direction. In order to control the direction of light irradiation from the light irradiation means, the light irradiation means may be rotatable with the use of a small-sized motor or the like. Alternatively, the light irradiation means may be provided with a prism or the like that is used for changing the direction of light irradiation. Then, such prism may be moved.

In addition, a part of a thin tube subjected to light irradiation may be provided with a light reflection material. In such case, the part exposed to reflected light is provided with an actuator. By controlling the position of such light reflection material and the position of the actuator, it becomes possible to allow the forward end of a thin tube to be flexed in a desired direction.

Also, the position of a part subjected to light irradiation can be changed. For instance, an optical fiber accommodated in a thin tube is moved in the anteroposterior direction in the thin tube such that light irradiation can be performed at an arbitrary position in the anteroposterior (longitudinal) direction of the light-absorbing material.

In addition, a thin tube that has been preliminarily flexed with an angle that causes an actuator at the forward end of the thin tube to be subjected to light irradiation upon light irradiation from the forward end may be used. In a case in which the degree of bending of a specific part of a lumen or the bending angle of a branching lumen is preliminarily known, a thin tube with a flexed forward end is inserted into such specific part, the position of an actuator is adjusted to be located at a position in the direction opposite to the traveling direction of the thin tube, and light irradiation is performed. Accordingly, it is possible to allow the forward end of the thin tube to be further flexed in a desired direction. As a result, it is possible to allow such thin tube to smoothly travel through a part having a large degree of bending. Also, in the case of a branching site, it is possible to allow such thin tube to travel to a desired branching part.

Further, by changing the intensity of light irradiation, the actuation level of the actuator can be changed. Thus, it is possible to control the degree of flection (namely, the flexed angle) of the forward end of a thin tube. For instance, in a case in which an actuator is a deformable material that is deformed by heat, the stronger the intensity of light irradiation, the larger the quantity of heat generated. Thus, the level of deformation of a deformable material is increased, resulting in flection of the forward end of a thin tube to a greater extent. In such case, the appropriate degree of flection of a thin tube can be determined by monitoring the degree of flection of the forward end of a thin tube before light irradiation. For instance, in a case in which a thin tube has a means of observing the inside of a lumen, such as an endoscope, it is possible to know the position of the thin tube and the degree of flection of the forward end of the thin tube with the use of such observation means. In addition, based on x-ray illumination images, it is possible to know the degree of flection of the forward end.

An example of a laser-generating device is LASER1-2-3 SCHWARTZ (ELECTRO-OPTICS).

Further, it is possible to use the apparatus of the present invention having a double lumen thin tube structure (comprising parent and child catheters). Such double lumen thin tube structure comprises an inner thin tube and an outer thin tube. Preferably, such double lumen structure ranges from the distal end part to the proximal end part of a double lumen thin tube. For instance, an inner thin tube may be provided inside a lumen of an outer a thin tube. In such case, the inner thin tube is provided with the aforementioned actuator that is operated via light irradiation. It is possible to move the inner thin tube in the anteroposterior direction (axial direction) by allowing it to slide within the outer thin tube. In addition, it is possible to rotate the inner thin tube within the outer thin tube. For instance, when a torque transmission tube is used as an inner thin tube, it becomes possible to rotate such inner thin tube. As described above, when the inner thin tube is moved in the anteroposterior direction and rotated, it is possible to move the actuator disposed inside the inner thin tube to a desired position. In such case, the outer thin tube may be moved without changes in the position of the inner thin tube.

The outer thin tube may be provided with the aforementioned device for sensing light irradiation. It is possible to detect the flexed direction of the outer thin tube with the use of such device. Then, the inner thin tube is moved in a manner such that the actuator located at the forward end of the inner thin tube is positioned opposite to the flexed direction. In such case, the device that has sensed light irradiation is located on an exterior side with respect to the flexed direction of the outer thin tube. Thus, a groove or paint is preliminarily provided on a thin tube in the longitudinal (axial) direction for marking such that the position of the device that has sensed light irradiation is confirmed. Also, the position of an actuator of the inner thin tube is confirmed in the same manner. Thus, it is possible to adjust the position of the actuator of the inner thin tube with the use of the aforementioned marking in a manner such that the actuator is located on the direction opposite to the flexed direction.

The apparatus of the present invention is used as described below. Herein, the drawings show examples of introducing a thin tube into a blood vessel.

First, a thin tube 1 of the present invention is inserted into a lumen with the use of a guide wire 8 or the like.

As shown in FIG. 1A, when a thin tube 1 reaches a bending part of the lumen, the thin tube 1 comes into contact with the wall of the lumen and the forward end of the thin tube is then lightly and passively flexed in the traveling direction of the lumen. However, in such case, if it is attempted to further insert the thin tube 1, it is impossible to smoothly do so. Accordingly, the tube becomes stuck or the wall of the lumen is damaged. Herein, the expression “passively flexed” indicates a situation in which a part of a thin tube comes into contact with a lumen so that the thin tube receives pressure and thus is flexed.

When the thin tube of the present invention reaches a bending site or an inner structure of a construct and comes into contact therewith so that the forward end of the thin tube is lightly and passively flexed, it becomes difficult to insert the thin tube so that the thin tube becomes stuck. At such time, a light-absorbing/deformable material 4 is irradiated with light in one embodiment of the use of the thin tube of the present invention (FIG. 1B). When the forward end of a thin tube 1 is not flexed, light 5 travels in parallel with the traveling direction of a thin tube 1 such that the inner wall of a thin tube 1 is not irradiated with the light 5 even if irradiation of light 5 is performed from an optical fiber 2 of the thin tube 1. However, when the forward end of a thin tube 1 is lightly flexed, the inner wall of a thin tube 1 is subjected to irradiation of light 5 in a straight direction from a light optical fiber 2, such inner wall being located opposite to the flexed side (FIG. 1B: right view). The right view of FIG. 1B is an enlarged view of the circle of the left view. In the apparatus of the present invention, a light-absorbing material and a deformable material come into contact with each other at a part irradiated with light 5 or they are integrated into a single material so as to exist at such part (light-absorbing/deformable material 4). A light-absorbing material absorbs light and generates heat so that the generated heat is conducted to a deformable material. The temperature of such deformable material increases due to the conducted heat so that the material becomes deformed and extended. Alternatively, a deformable material itself serves as a light-absorbing material. In such case, a deformable material absorbs light and generates heat so as to be deformed and extended. When a deformable material is a bimetal, a material having a large thermal expansion rate is disposed outside a thin tube 1 and a material having a small thermal expansion rate is disposed inside a thin tube 1. Thus, upon heat conduction, the material disposed outside of a thin tube 1 is expanded (extended) more than the material disposed inside thereof such that the bimetal is bent and the forward end of a thin tube 1 is flexed to the side opposite to the side at which the bimetal exists. Accordingly, the forward end of a thin tube 1 is allowed to be further actively flexed to the side to which the tube has been passively flexed due to contact with a lumen. In a case in which a deformable material is a shape-memory alloy, such material attempts to recover its original state (namely, the state of being extended). Thus, the forward end of a thin tube 1 is further allowed to be actively flexed to the side to which the tube has been passively flexed due to contact as described above. At such time, a thin tube 1 is further inserted so that the thin tube 1 travels in the flexed direction. Thus, in the apparatus of the present invention, when the forward end of a thin tube 1 is lightly flexed, it is possible to allow a thin tube 1 to be further flexed to the flexed direction. Also, the apparatus of the present invention is an apparatus whereby the interior side of a flexed thin tube 1 and the exterior side of a flexed thin tube 1 that has been flexed are automatically discriminated from each other so that it is possible to allow the tube to be further flexed to the interior side. Specifically, the apparatus of the present invention involves a thin tube that is allowed to be flexed in the traveling direction by detecting the traveling direction.

The position of a light irradiation section is not limited. However, such position is preferably located behind the forward end of a thin tube 1. In such case, even if the forward end of a thin tube 1 is lightly flexed, the irradiation direction is the direction in which the tube was disposed before reaching a bending part. If light irradiation is performed in such state, a light-absorbing material or a deformable material having light-absorbability (light-absorbing/deformable material 4), which is disposed on the side opposite to the flexed side of a thin tube 1, is irradiated with light 5 as shown in FIG. 1B. The position of a light irradiation section is variable. Thus, when a light-transmitting fiber 2 is moved in the anteroposterior (longitudinal) direction in a thin tube 1, such light irradiation section varies so that the part subjected to light irradiation also varies. In a case in which a plurality of light-absorbing materials or deformable materials having light absorbability (light-absorbing/deformable materials 4) are provided on a thin tube 1 in the longitudinal direction, a thin tube 1 is allowed to be flexed at a desired position.

In addition, in another embodiment of the use of the present invention, a light-absorbing/deformable material 4 may be irradiated with light 5 after the forward end of a thin tube 1 is lightly flexed before the thin tube comes into contact with the inner wall of the flexed part of a lumen. In such case, the forward end of a thin tube 1 is further flexed based on the principle described above. The forward end can be flexed before coming into contact with a lumen by a conventional method for allowing a catheter flexed.

Further, in another embodiment of the use of the present invention, when the direction of irradiation of light 5 is changed, a light-absorbing/deformable material 4 disposed on a desired side of the forward end of a thin tube 1 is extended such that the forward end of a thin tube 1 is allowed to be flexed in a desired direction. As described above, when the forward end of a thin tube 1 is allowed to be flexed in a desired direction, it is possible to concentrically control the central axis of a lumen and the axis of a thin tube. Thus, a thin tube 1 can always be directed to the center of a lumen. As descried above, when a thin tube 1 is controlled to be directed to the center of a lumen, clear images can always be obtained upon endoscopic observation. Also, in cases of angioplasty and the like, it is possible to guide an apparatus used for angiogenesis to an appropriate position. In such case, it is necessary to monitor the position of the forward end of a thin tube 1 for positional control. However, for instance, a marker that can generate x-rays is allowed to bind to the forward end of a thin tube such that the x-rays generated may be monitored from the outside. In cases of endoscopes and the like, monitoring may be carried out by observing images obtained via an endoscope. A thin tube into which an endoscopic apparatus has been incorporated will be described below.

In addition, the apparatus having a double lumen thin tube structure of the present invention is used as described below.

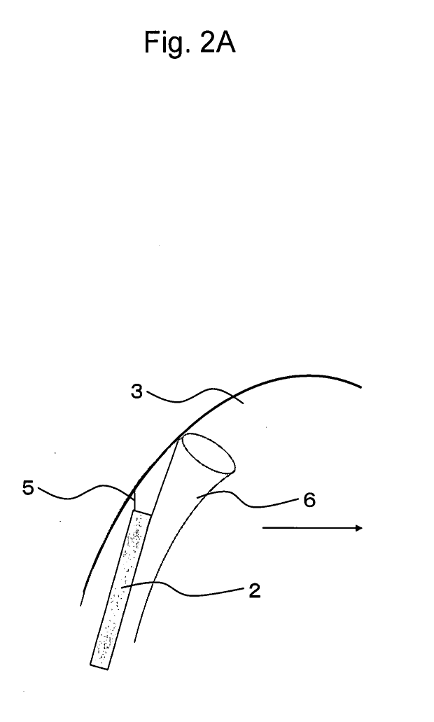

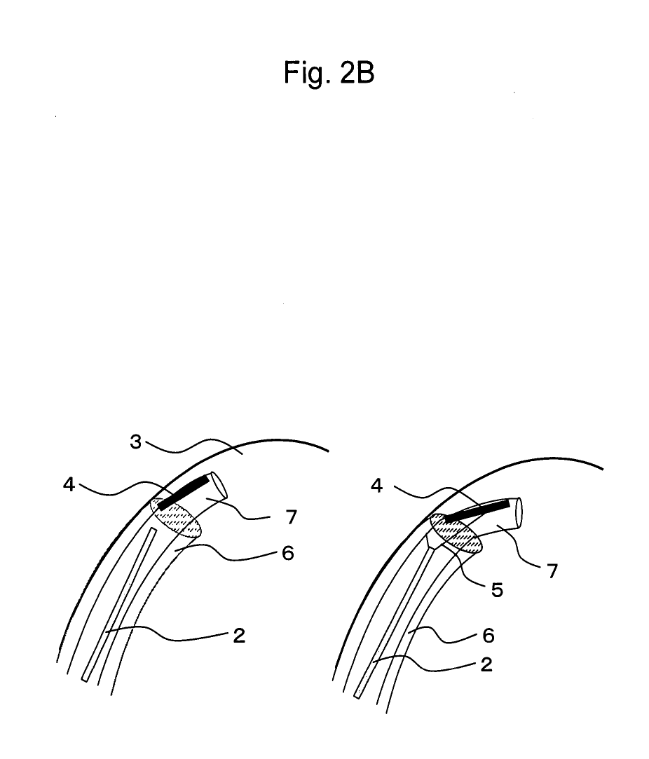

As shown in FIG. 2A, when an outer thin tube 6 reaches a bending part of a lumen, the outer thin tube 6 comes into contact with the wall of the lumen so that the forward end of the thin tube is lightly and passively flexed in the traveling direction of the lumen. A device for sensing irradiation of light 5 is provided covering the whole circumference of the forward end of the outer thin tube 6. Thus, upon irradiation of light 5, such device that is disposed on the side opposite to the flexed direction senses light. Then, it is judged that the outer thin tube 6 is flexed in a direction opposite to the side subjected to light irradiation so that the flexed direction can be detected. Note that the drawing of the inner thin tube is omitted in FIG. 2A.

In such case, an outer thin tube 6 is exclusively inserted into a lumen and the thin tube 6 is then placed at a bending site or a branching site. Subsequently, an inner thin tube 7 may be inserted thereinto (FIG. 2B: left view). For instance, positions of a branching part and a sharp bending part of a blood vessel are known in advance. Thus, when a thin tube is allowed to pass through such part, an outer thin tube 6 is first inserted and placed at such part and the forward end of an inner thin tube 7 is then allowed to reach the site while passing through inside the outer thin tube 6. Then, the forward end of the thin tube 7 is allowed to be flexed in a desired direction so that it becomes possible to allow the inner thin tube 7 to pass through a bending site or to pass through a branching site in a desired branching direction. Further, in such case, an inner thin tube 7 having a forward end that has preliminarily been flexed at a certain angle may be used. Such inner thin tube 7 having a forward end that has preliminarily been flexed at a certain angle is adjusted so as to be located in a manner such that a part at which a light-absorbing/deformable material 4 has been provided is located on the exterior side of the flexed site, followed by irradiation of light 5. Thus, the forward end of the inner thin tube 7 is allowed to be further flexed in a desired direction. Thus, it is possible to allow such thin tube to pass through a sharp bending site and a branching site.

Next, the inner thin tube 7 is moved in the anteroposterior direction and rotated such that the position of a light-absorbing/deformable material 4 (actuator) that has been provided to the thin tube 7 is adjusted to a position at which the material is irradiated with light (FIG. 2B: right view). When the deformable material is irradiated with light in such state, the deformable material is deformed so that the inner thin tube 7 is further flexed in the direction in which the outer thin tube 6 has been flexed.

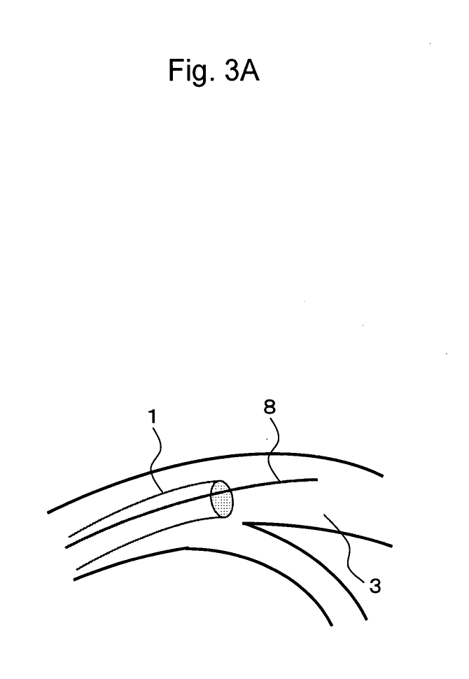

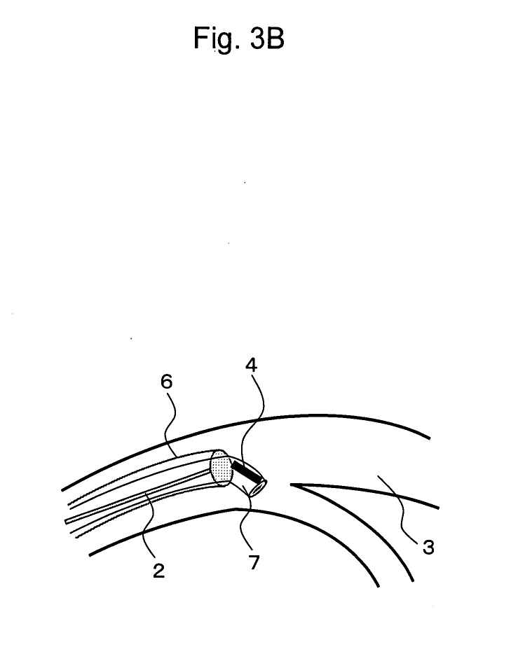

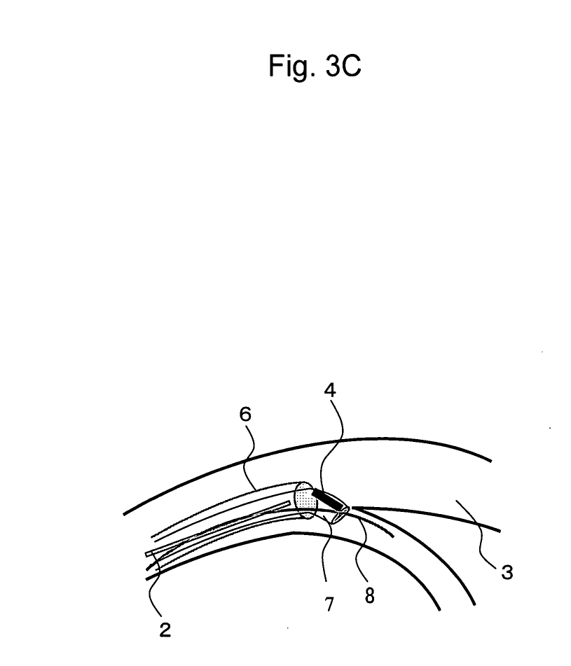

With the use of the apparatus of the present invention, the traveling direction of a thin tube is controlled at a branching site of a lumen such as a branching site of a blood vessel in the manner described below. FIG. 3A shows a conventional method for inserting a catheter into a blood vessel. In FIG. 3A, only a guide wire 8 is inserted into a blood vessel. As shown in FIG. 3A, a guide wire 8 is likely to be inserted into an exterior blood vessel 3 having a large curvature radius.

Meanwhile, FIG. 3B shows the thin tube of the present invention, which has a double lumen thin tube structure. In FIG. 3B, a deformable material (light-absorbing/deformable material 4) is applied to an inner thin tube 7. An outer thin tube 6 is placed at a branching site of a blood vessel in advance. In such case, the position of the outer thin tube 6 may be monitored by a conventional method. For instance, in a case in which such thin tube has a means of observing the inside of a lumen, such as in the case of an endoscope, it is possible to know the position of the thin tube and the degree of flection of the forward end of the thin tube with the use of such observation means. In addition, with the use of x-ray illumination images, it is possible to know the degree of flection of the forward end. Subsequently, an inner thin tube 7, the forward end of which has a flection mechanism, is inserted into the outer thin tube 6 that has preliminarily been placed, the forward end is allowed to protrude from the outer thin tube, and the forward end is subjected to light irradiation from an optical fiber 2 that is disposed in the vicinity of the forward end of the inner thin tube having a flection mechanism. Accordingly, a light-absorbing/deformable material 4 on the outside of curvature of the thin tube is irradiated with light 5 and the deformable material is extended by light or heat, so that the flexible thin tube is flexed to the inside of curvature of a blood vessel. Then, the thin tube in such state is further inserted so that it is possible to insert a thin tube in a desired direction.

In addition, as shown in FIG. 3C, when a guide wire 8 is inserted into a thin tube that has been flexed, it is possible to insert a guide wire 8 into a branching blood vessel having a large degree of curvature. Thereafter, the thin tube may travel along with the guide wire.

The present invention encompasses a method for operating the forward end of the aforementioned thin tube or double lumen thin tube in a lumen.

The thin tube for medical use of the present invention can be applied to a variety of remedies.

For instance, the thin tube for medical use of the present invention is used as an endoscope such that the inside of a lumen of a living body can be observed. In addition, the thin tube for medical use of the present invention can be used for endoscopic surgery or the like.

The thin tube for medical use of the present invention may comprise a variety of means depending on the purposes thereof. In one case, for instance, an outer thin tube of the aforementioned double lumen thin tube may accommodate a variety of means in addition to an inner thin tube. In such case, the thin tube of the present invention that can be flexed has a function of flection in its entirety and other means have functions for treatment and the like.

For instance, the thin tube for medical use of the present invention may comprise a means of administration of a PDT agent for carrying out photodynamic therapy (PDT), a means of irradiation of high-intensity pulsed light such as a laser, and a means of electrical transmission of high-intensity pulsed light. In addition, the thin tube for medical use of the present invention may comprise a Rotablator, cutter, or the like used for arteriosclerosis therapies.

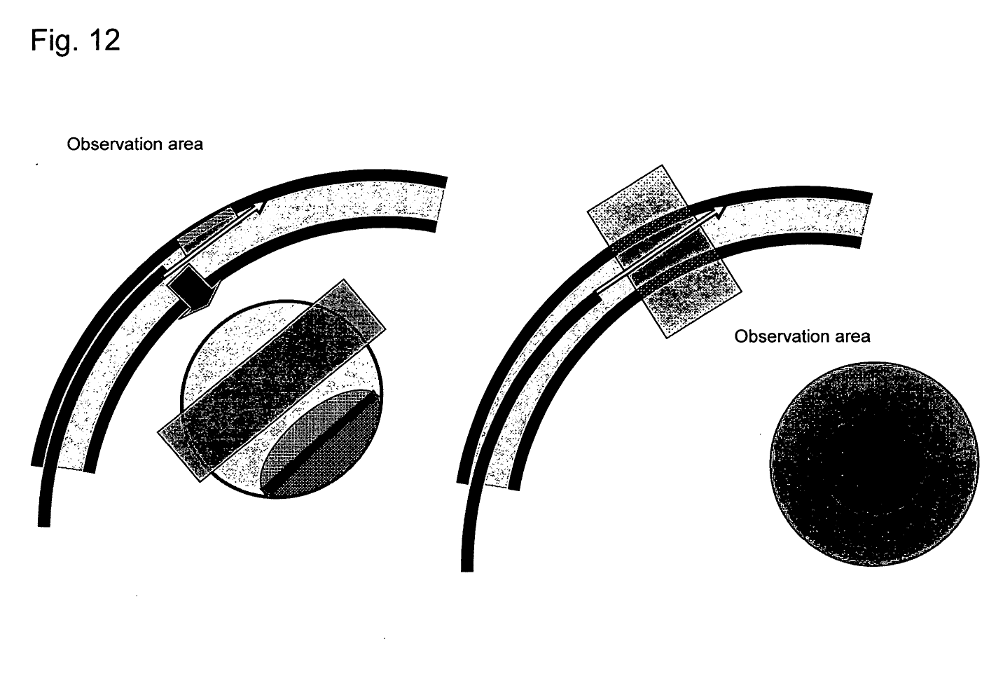

Further, the thin tube of the present invention may be used by incorporating thereinto an endoscope apparatus comprising a high-intensity pulsed light generating means and a high-intensity pulsed light transmitting means for transmitting high-intensity pulsed light, such endoscope irradiating the inside of a lumen with high-intensity pulsed light and generating vapor bubbles so as to be able to temporarily exclude liquid in the lumen. Preferably, such endoscope apparatus is an angioscope apparatus that can temporarily exclude blood in a blood vessel. Hereafter, an angioscope used for observation of the inside of a blood vessel will be described. However, the thin tube of the present invention into which an endoscope apparatus has been incorporated can be used for observation of any type of lumen filled with liquid in addition to a blood vessel. When blood in a blood vessel is excluded (that is to say, when the inside of a blood vessel is cleared of blood with the use of gas), a visual space with little scattering can be obtained so that it becomes possible to clearly observe the surface of the blood vessel due to surface reflections. In addition, it becomes possible to allow an observation image to have a high field angle so that a strong spatial effect can be obtained. Further, also in a case in which illumination light with the same level of light intensity is used, an illumination angle is increased and a surface reflection rate is increased, compared with a case in which vapor bubbles are not generated. Thus, it is possible to further illuminate the inside of a blood vessel to be observed so that an improved high-precision image can be obtained. In such case, an endoscope apparatus is provided in the thin tube of the present invention, the forward end of which can be flexed, in a manner such that an observation means is provided at the forward end. Then, the forward end of the thin tube is allowed to be flexed in a direction for observation so that observation in such direction can be carried out. For instance, in a case in which an thin tube into which an endoscope apparatus has been incorporated is inserted into a blood vessel, when the forward end of the thin tube comes into contact with the inner wall of the blood vessel at a bending site of the blood vessel, the observation means of the endoscope is not directed toward the inside of the blood vessel, so that it is impossible to sufficiently observe the inside of the blood vessel. In such case, irradiation with laser light is performed so as to allow the forward end of a thin tube to be flexed such that the forward end of a thin tube is directed toward a direction in which the deep interior of the blood vessel can be observed. At such time, vapor bubbles may be generated for observation. FIG. 12 shows observation of the inside of a blood vessel with the use of the thin tube of the present invention into which an angioscope had been incorporated. The left view of FIG. 12 shows a thin tube inserted into a blood vessel, with the forward end of the thin tube coming into contact with the inner wall of the blood vessel. Under such condition, an observation means such as an endoscope cannot be directed to the deep interior of a blood vessel. Accordingly, only an image of the wall of a blood vessel with which a thin tube has come into contact can be observed as shown in the left circle of FIG. 12. When the forward end of a thin tube comes into contact with the inner wall of a blood vessel, a light-absorbing/extensible material of the thin tube is irradiated with laser light. Accordingly, the forward end of the thin tube is flexed such that it is directed to the deep interior of the blood vessel. The right view of FIG. 12 shows such condition. As shown in the right circle of FIG. 12, a clear image showing the deep interior of a blood vessel can be obtained for observation when a thin tube is directed to the center of the blood vessel. In such case, it is important that the forward end of a thin tube can be confirmed to be accurately directed to the deep interior of a blood vessel. With the use of the thin tube of the present invention, it is possible to determine the degree of flection of the thin tube. Also, it is possible to allow the forward end of the thin tube to be flexed in a desired direction. Thus, it is possible to accurately confirm the direction in which a thin tube is directed. In addition, when the inside of a blood vessel is observed, the central axis of a blood vessel and that of a thin tube into which an endoscope apparatus has been incorporated are allowed to overlap each other. In other words, they are allowed to concentrically overlap each other so as to obtain a concentric view. Thus, the deep interior of a blood vessel may be observed after securing an all-around concentric view. Alternatively, the wall of a specific site of a blood vessel may be exclusively targeted for observation. Depending on which part of a blood vessel wall is observed, the flexed direction and the degree of flection of the forward end of a thin tube may be changed. In the case of a usual endoscope, when the concentricity between an endoscope and a blood vessel is improved (that is to say, when their central axes are allowed to overlap each other), illumination light that can be used for irradiation from the endoscope travels in a single direction. Thus, the center of a image observed becomes significantly dark. Meanwhile, in a case of an endoscope apparatus comprising a high-intensity pulsed light generating means and a high-intensity pulsed light transmitting means for transmitting high-intensity pulsed light, such endoscope irradiating the inside of a lumen with high-intensity pulsed light and generating vapor bubbles so as to be able to temporarily exclude liquid in the lumen, the surface reflection inside a blood vessel to be observed becomes large. Thus, it becomes possible to illuminate the entire observation site with the use of diffuse reflection.

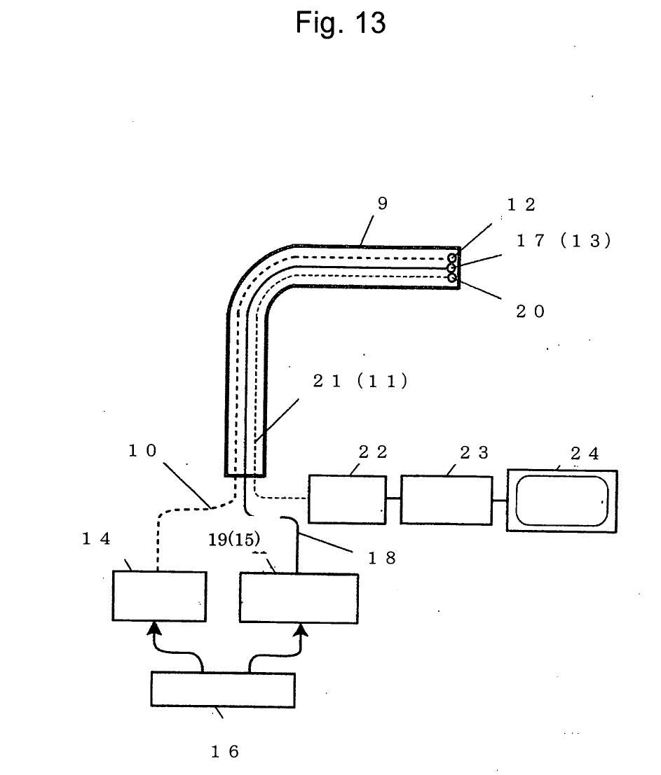

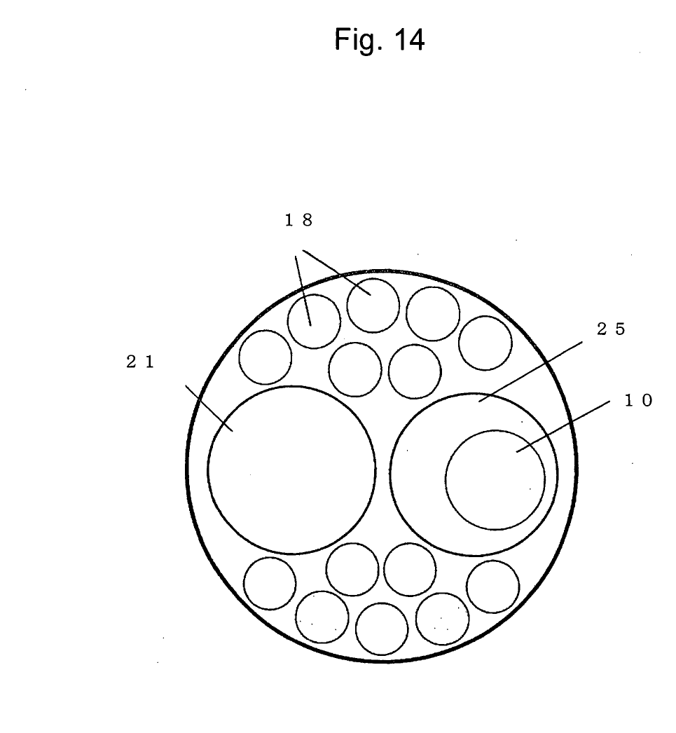

The aforementioned endoscope apparatus is described in WO2005/063113 in detail. In addition, FIG. 13 shows the endoscope apparatus. FIG. 14 shows a cross-sectional view of a catheter part of the endoscope apparatus.

In the case of the thin tube of the present invention, the forward end of which can be flexed, and into which an endoscope apparatus comprising a high-intensity pulsed light generating means and a high intensity pulsed light transmitting means for transmitting high intensity pulsed light has been incorporated, such endoscope irradiating the inside of a lumen with high-intensity pulsed light and generating vapor bubbles so as to be able to temporarily exclude liquid in the lumen, the forward end of a catheter 9 shown in FIG. 12, for example, may be provided with a device for sensing light irradiation and/or an actuator that is operated via light irradiation. In addition, the catheter 9 may be provided therein with a light transmission means whereby the aforementioned device and/or actuator are/is irradiated with light. Such light transmission means may be connected to, for example, a high intensity pulsed light source 14 such that light with which the aforementioned device and/or actuator is irradiated is generated from such light source. Alternatively, a light source exclusively used for light irradiation may be separately used. Further, in a case in which a thin tube is used as an inner thin tube and an outer thin tube is provided outside the inner thin tube, it is possible to obtain the double lumen thin tube of the present invention into which an endoscope apparatus comprising a high-intensity pulsed light generating means and a high-intensity pulsed light transmitting means for transmitting high-intensity pulsed light has been incorporated, such endoscope irradiating the inside of a lumen with high-intensity pulsed light and generating vapor bubbles so as to be able to temporarily exclude liquid in the lumen.

### EXAMPLES

The present invention is hereafter described in greater detail with reference to the following examples, although the technical scope of the present invention is not limited thereto.

### Example 1

### Experiment of Allowing a Thin Tube Flexed

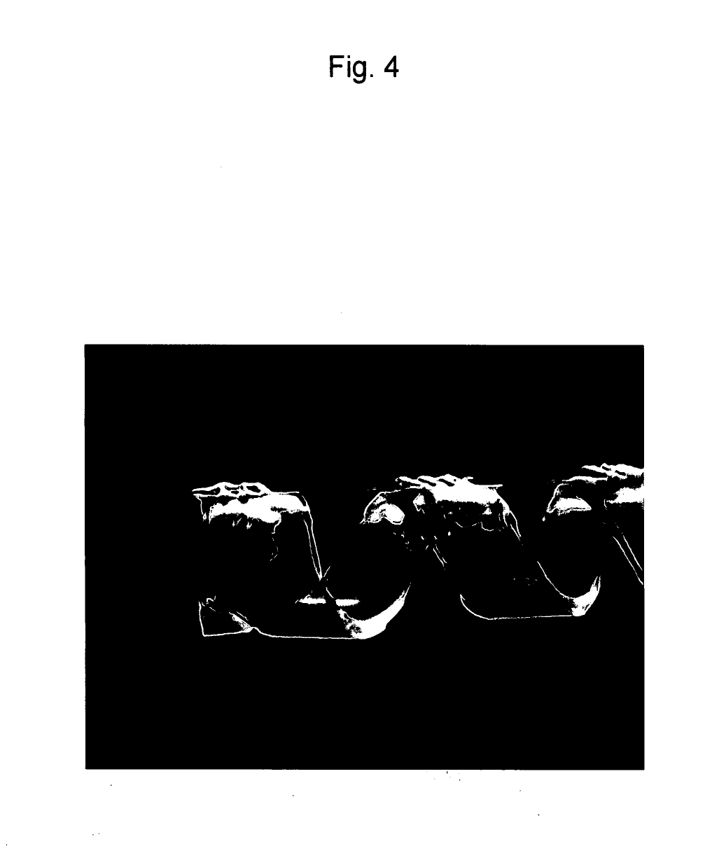

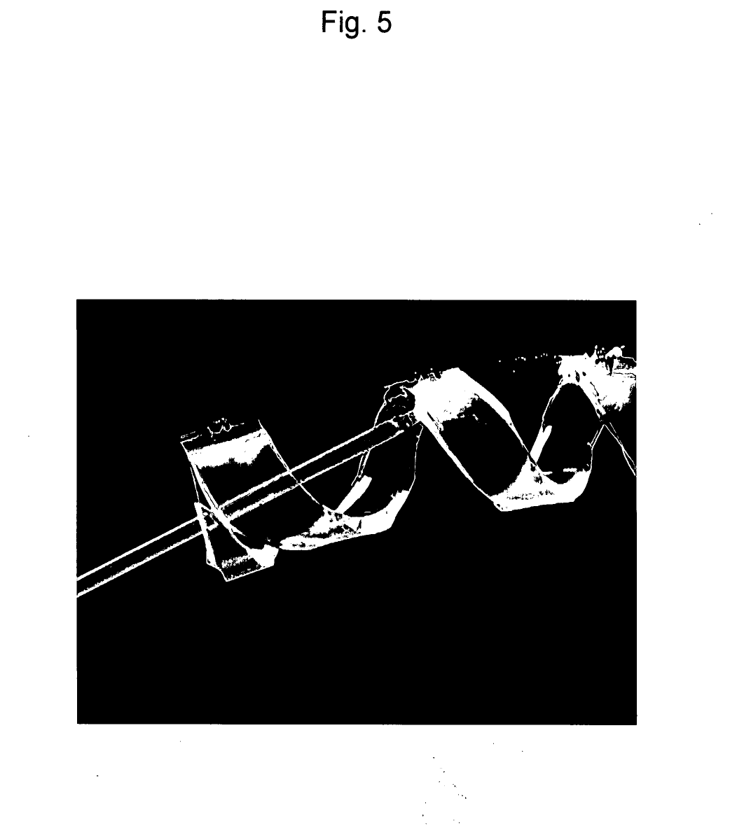

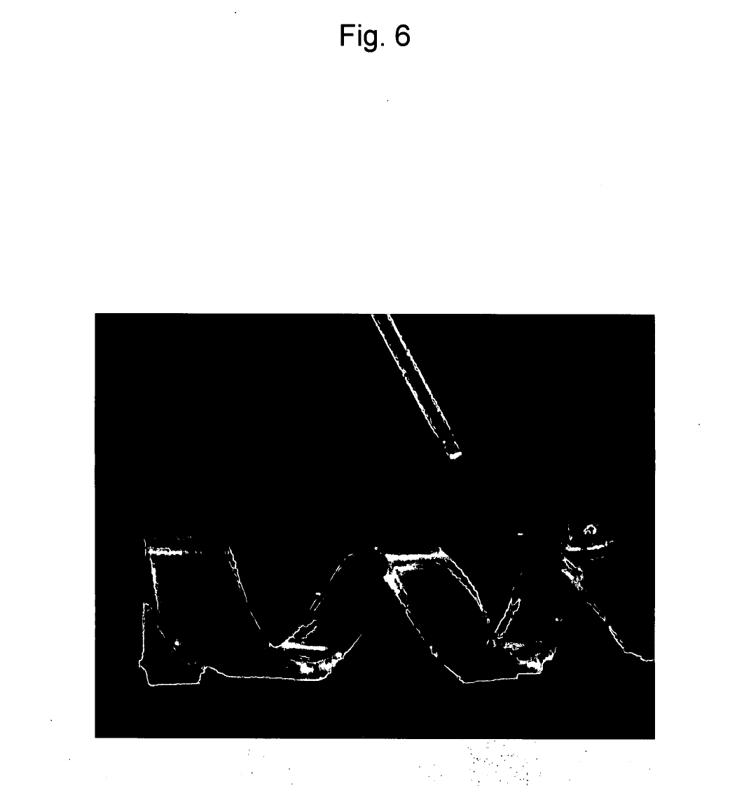

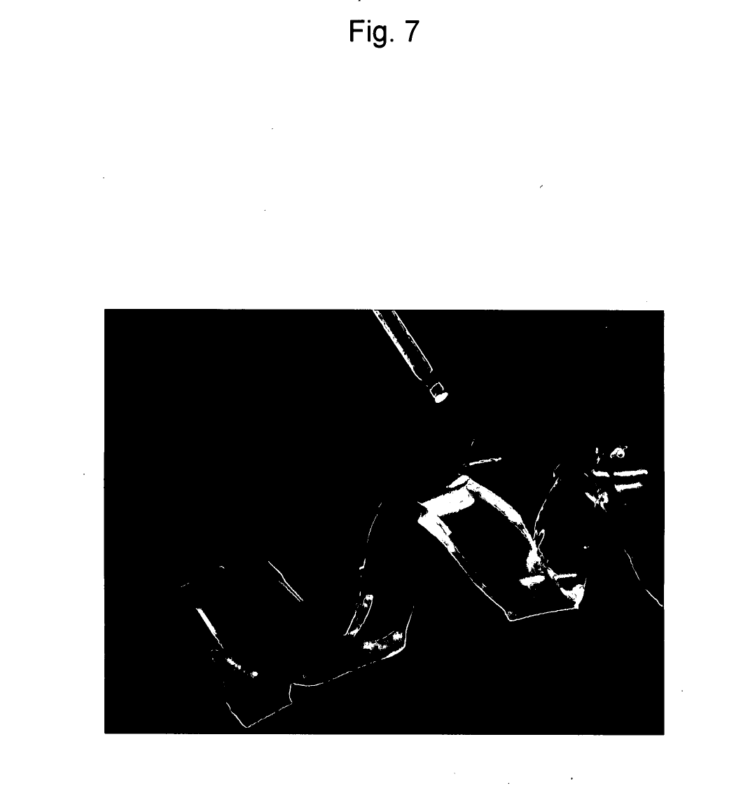

A tube 9 mm in inner diameter (Sanyo Rikagaku Kikai Seisakusho) was spirally coiled. A bimetal was attached to a single site on the outer side of the tube. The bimetal used was BR-1 (size: 4 mm×60 mm, NEOMEX). The bimetal was attached in a manner such that the high-expansion metal thereof was disposed on the outside of the tube. Irradiation with a semiconductor laser (3 W) was conducted from the inside and outside of the tube. The laser-generating device used was UDL-60 (OLYMPUS). FIGS. 4 and 5 show experiments of laser irradiation from the inside of the tube. FIGS. 6 and 7 show experiments of laser irradiation from the outside of the tube. FIGS. 4 and 6 show the tube before laser irradiation. FIGS. 5 and 7 show the tube immediately after laser irradiation. In FIGS. 4 to 7, the bimetal is shown as a stick attached to the upper part of the tube. In FIGS. 5, 6, and 7, a tube-like thin stick is an optical fiber used for laser irradiation. As shown in FIGS. 5 and 7, the tube is flexed as a result of laser irradiation on the bimetal.

### Example 2

### Experiment of Allowing a Thin Tube Flexed in a Lumen

A tube 38 mm in inner diameter was used as a simulated blood vessel. The simulated blood vessel was curved so as to be immobilized in that state. Then, an experiment similar to that conducted in Example 1 was carried out in the simulated blood vessel.

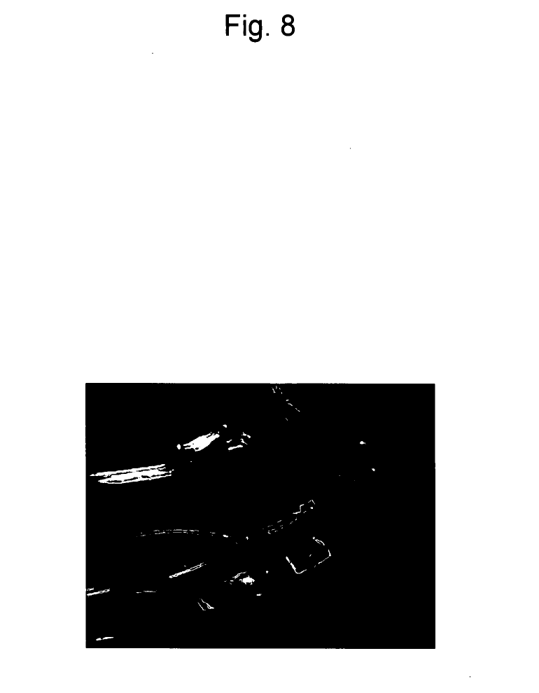

The tube 9 mm in inner diameter to which a bimetal had been attached used in Example 1 was inserted into the simulated blood vessel in a curved state. The tube was allowed to be flexed in a direction parallel to the direction of the curvature of the simulated blood vessel (FIG. 8).

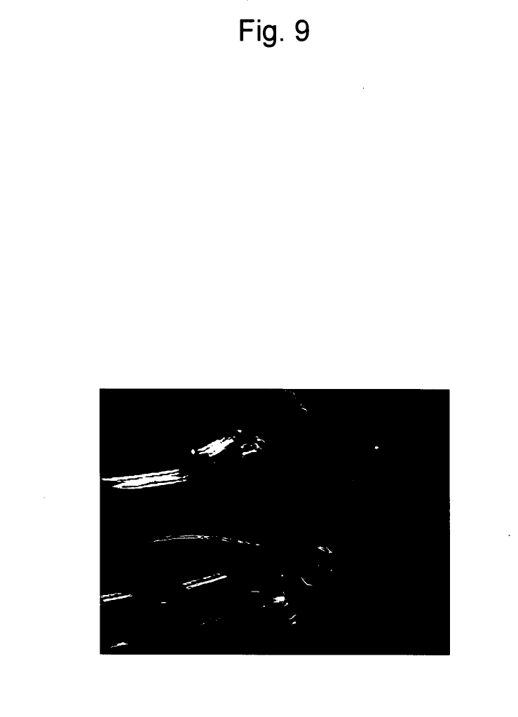

Under such condition, irradiation with a semiconductor laser (3.5 W) from a fiber 750 μm in inner diameter and 1 mm in outer diameter that had been inserted into the tube was performed. FIG. 9 shows results of the irradiation. As shown in FIG. 9, the tube to which a bimetal had been attached further flexed in a direction identical to the bending direction of the simulated blood vessel. The result indicates that the forward end of the thin tube of the present invention is allowed to be actively flexed to the side to which the thin tube has been passively flexed upon light irradiation.

### Example 3

### Experiment of Measuring the Temperature of a Tube

Upon irradiation with a semiconductor laser (3 W) from a fiber 750 μm in inner diameter and 1 mm in outer diameter that had been inserted into a tube 9 mm in inner diameter, temperatures of the following sites were measured using a thermocouple (TS-T-36-1, Ishikawa Trading Co., Ltd.). The measurement of temperature was performed at the site at which laser irradiation was performed, a site located at a distance of ¼ of the circumference of the tube from the irradiation site, and a site located at a distance of ½ of the circumference of the tube from the irradiation site.

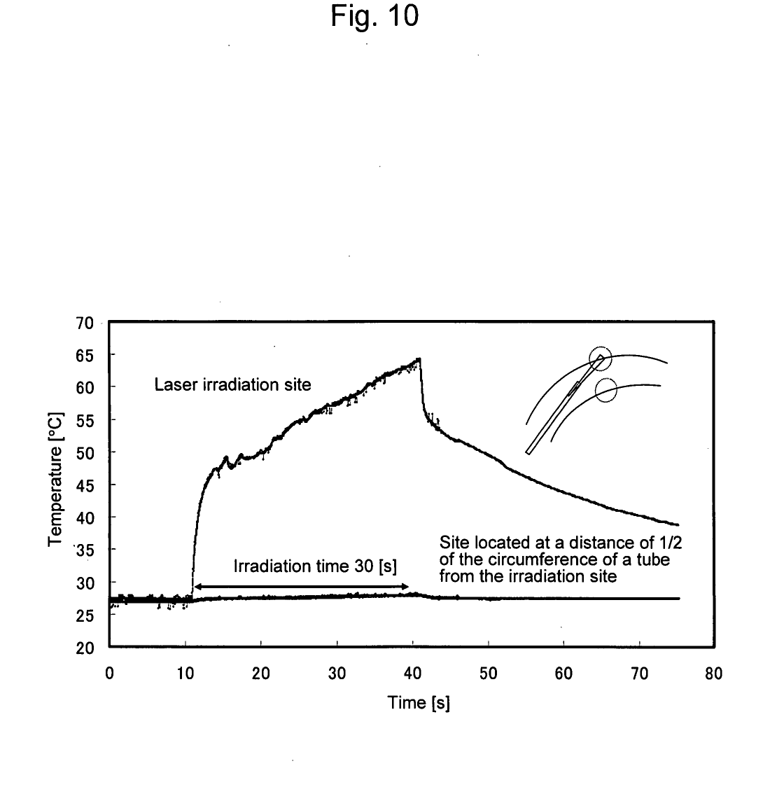

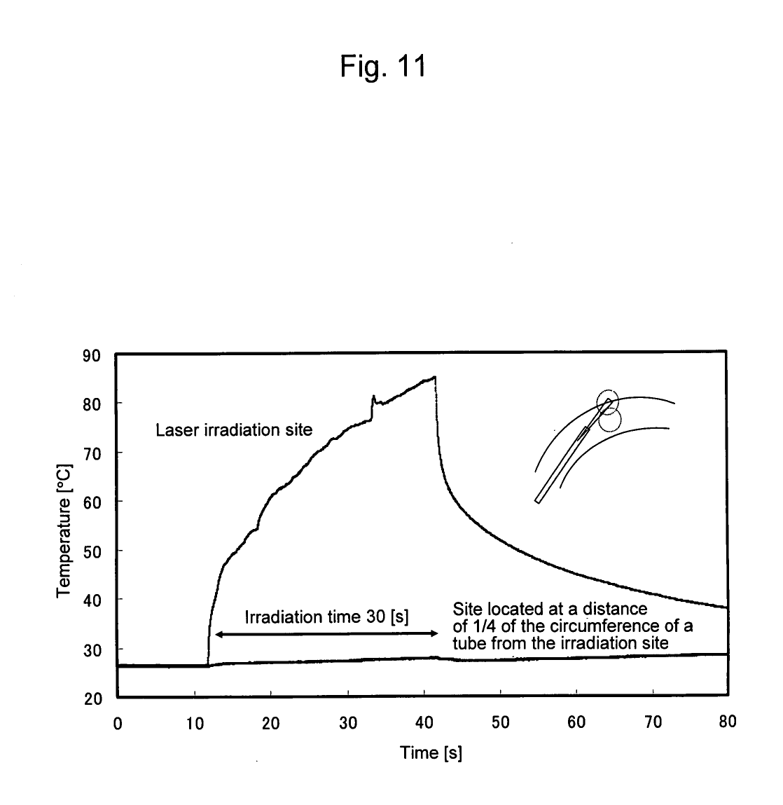

FIG. 10 shows results of the measurement obtained at the site of laser irradiation and at the site located at a distance of ½ of the circumference of the tube from the irradiation site. FIG. 11 shows results of the measurement obtained at the site of laser irradiation and at the site located at a distance of ¼ of the circumference of the tube from the irradiation site. As shown in FIGS. 10 and 11, temperature increase was observed at the site of laser irradiation, while temperature increase was not substantially observed at the site located at a distance of ¼ of the circumference of the tube from the irradiation site or at the site located at a distance of ½ of the circumference of the tube from the irradiation site. These results indicate that a site subjected to laser irradiation can be determined by measuring temperature increase at each site of a thin tube. Thus, when a light-absorbing material/extensible material exists at a site subjected to laser irradiation, it is understood that a tube became flexed at such site. FIG. 10 and the upper right drawing in FIG. 11 show conditions of laser irradiation.

### Example 4

### Examination of an Endoscope Apparatus Comprising a High-Intensity Pulsed Light Generating Means and High-Intensity Pulsed Light Transmitting Means for Transmitting High-Intensity Pulsed Light, Such Endoscope Irradiating the Inside of a Lumen with High Intensity Pulsed Light and Generating Vapor Bubbles so as to be Able to Temporarily Exclude Liquid in the Lumen

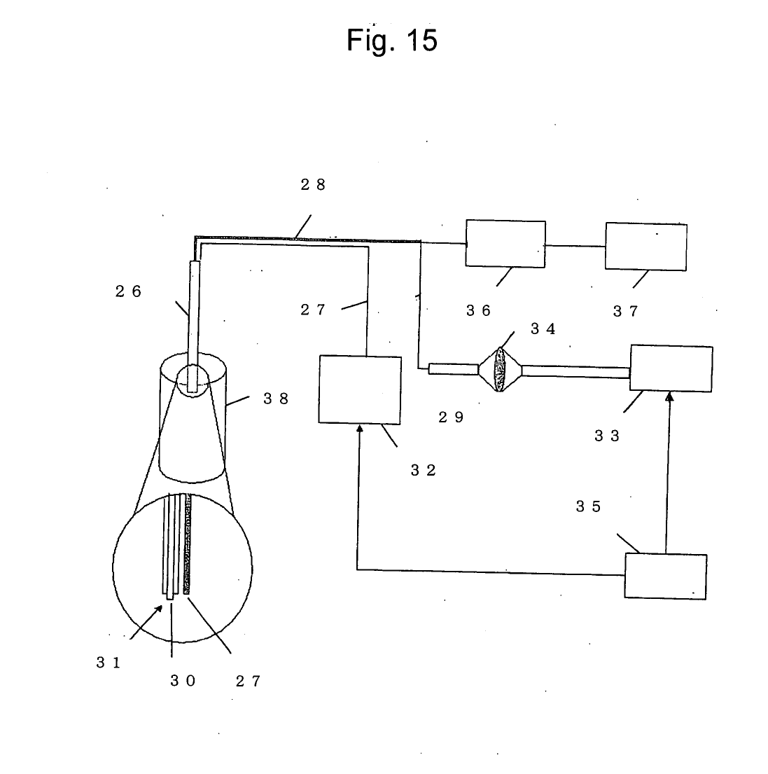

The endoscope used in this Example is shown in FIG. 15. As shown in FIG. 15, a small-diameter endoscope 30 was installed in a stainless steel sheath 31 having a length of approximately 3 cm and an inner diameter of 0.8 cm.

An image guide 28 and a light guide 29 were placed in the small-diameter endoscope 30. A laser transmission fiber 27 was disposed along the endoscope and these were placed in a catheter sheath 26. In this case, the small-diameter endoscope 30, (i.e., the distal ends of the image guide 28 and light guide 29) was made to slightly protrude from the laser transmission fiber 27. Identical quartz optical fibers were used as optical fibers for image pickup in the laser transmission optical fiber 27 and the image guide 28. A plastic light guide was used as the light guide 29. The diameter of the laser transmission fiber 27 was approximately 0.6 mm and the diameter of the small-diameter endoscope 30 in which the light guide 29 and image guide 28 had been integrated together was approximately 0.7 mm. The laser transmission optical fiber 27 was connected to a Ho:YAG laser generator 32 (LASER1-2-3SCHWARTZ (ELECTRO-OPTICS (U.S.A.))). Several fibers were used as optical fibers for transmission of pulsed illumination light from the light guide 29 for pulsed light illumination. Each optical fiber for transmission of pulsed illumination light was connected to a flash lamp 33 (fiber video flash MODEL FA-1J10TS (Nisshin Electronic Co., Ltd.)) through a condensing lens 34. In FIG. 15, thick white lines on both sides of the condensing lens 34 denote light. The above Ho:YAG laser generator 32 and flash lamp 33 were connected via a delay generator 35 (digital delay generator BNC555 Series (Seki Technotolon Corp.)). A SELFOC lens was disposed at the distal end of the optical fiber of the image guide 28 and the opposite end thereof was connected to a CCD camera 36 (endoscope 3CCD video camera system MV-5010A (Machida Endoscope Co., Ltd.)). Furthermore, the CCD camera 36 was connected to a monitor 37 (PVM-9040 (SONY)) via an RGB cable. Thus, it became possible to observe an image of an intravascular lumen with the use of the monitor 37.

The excised pig coronary artery and the pig blood vessel used in this Example were purchased from the Metropolitan Central Wholesale Market Meat Market. The pig coronary artery 38 was cut into pieces of approximately 5 cm for use. An end of the pig coronary artery 38 was ligated and physiological saline or heparinized pig blood was introduced thereinto. The distal end part of the catheter sheath 26 into which the aforementioned laser transmission optical fiber 27, light guide 29, and image guide 28 had been disposed was put in the saline or pig blood, followed by illumination with pulsed illumination light having a pulse width of 10 μs without laser irradiation. Then, images of the intravascular lumen taken by the CCD camera 36 were displayed on the monitor 37 and recorded via video. Further, the piece of the above pig coronary containing the pig blood was irradiated with laser so as to produce vapor bubbles. Then, images thereof were taken. The intensity of laser at such time was approximately 200 mJ/pulse and pulse width was approximately 200 μs. The images of the intravascular lumen that had been delayed by the delay generator and obtained by the CCD camera were displayed on the monitor and recorded via video.

When an image of the pig coronary artery into which pig blood had been introduced was taken without laser irradiation, the presence of the blood caused the entire image to become red and it was impossible to see the intravascular lumen. On the other hand, when transparent physiological saline was introduced into the pig coronary artery, it was possible to observe the intravascular lumen. In addition, when the blood was introduced into such artery and the artery was irradiated with a laser so as to generate vapor bubbles, the blood in the anterior portion of the catheter was temporarily excluded by the vapor bubbles, and thus it was possible to observe the intravascular lumen. The experiment of the artery containing saline imitated an endoscope test with a flushing liquid injected according to a conventional method. It was proven that it is possible to obtain images of an intravascular lumen with the use of the angioscope using high-intensity pulsed-light-induced bubbles of the present invention in the same way as in a conventional endoscope test wherein observation is performed by injecting a flushing liquid.

### Example 5

### Examination 2 of an Endoscope Apparatus Comprising a High-Intensity Pulsed Light Generating Means and a High-Intensity Pulsed Light Transmitting Means for Transmitting High Intensity Pulsed Light, Such Endoscope Irradiating the Inside of a Lumen with High-Intensity Pulsed Light and Generating Vapor Bubbles so as to Allow Temporary Exclusion of Liquid in the Lumen

A silicone tube was filled with milk. The inside of a lumen of the tube was irradiated with high-intensity pulsed light so as to generate vapor bubbles therein. Then, the inner wall of the tube was observed using an endoscope apparatus capable of temporarily excluding liquid in the lumen. The endoscope apparatus used was the same as that of Example 4. A silicon tube having an inner diameter of 3 mm was cut open, a piece of paper colored with water-resistant red ink was pasted inside of the tube, and the silicon tube was closed again. Next, a distal end part of a catheter sheath 26 of an endoscope apparatus, in which a laser transmission optical fiber 27, light guide 29, and image guide 28 had been disposed, was inserted into the silicon tube and the tube was put into milk such that the tube was filled with the milk. Next, irradiation with a pulsed laser was performed so as to generate vapor bubbles and images were taken. The laser intensity at such time was 200 mJ/pulse or 450 mJ/pulse at the end of the laser irradiation fiber. The pulse width was approximately 200 μs. The images of the intravascular lumen that had been delayed by a delay generator and obtained by a CCD camera were displayed on a monitor and recorded via video. The delay time was 70 μs or 140 μs when the laser intensity was 200 mJ/pulse and was 70 μs, 105 μs, 140 μs, 175 μs, or 210 μs when the laser intensity was 450 mJ/pulse. As a control for this case, images were taken without laser irradiation. Moreover, images of the tube filled with air but not with milk were taken as described above and they were designated as controls (in the air). When the laser intensity was 450 mJ/pulse, the size and brightness of an image of the inside of the silicon tube (a part that looks bright) taken at a different delay time were measured and expressed as relative values with respect to the values (each determined to be 1) at a delay time of 70 μs. The sizes of such images increase when a scattering liquid (milk) is located in front of a focus position because the images become out of focus. Meanwhile, the sizes of the images decrease when the scattering liquid (milk) is excluded to a site separate from the focus position because the focused images are obtained. Furthermore, the brightness of such screen indicates the extent to which the scattering liquid (milk) exists in the visual field for observation (a part that can be observed with illumination light) and the fact of getting dark indicates that the scattering liquid in the visual field for observation has been excluded. The images obtained were expressed using color-processing software (Photoshop (Adobe Systems, Inc., U.S.A.)) with an L*a*b* display system. The sizes of the images were obtained by measuring the radii of parts of Lab images each having a brightness of 20 or greater with the use of calipers. Such brightness was obtained by measuring the brightest part of the Lab images.

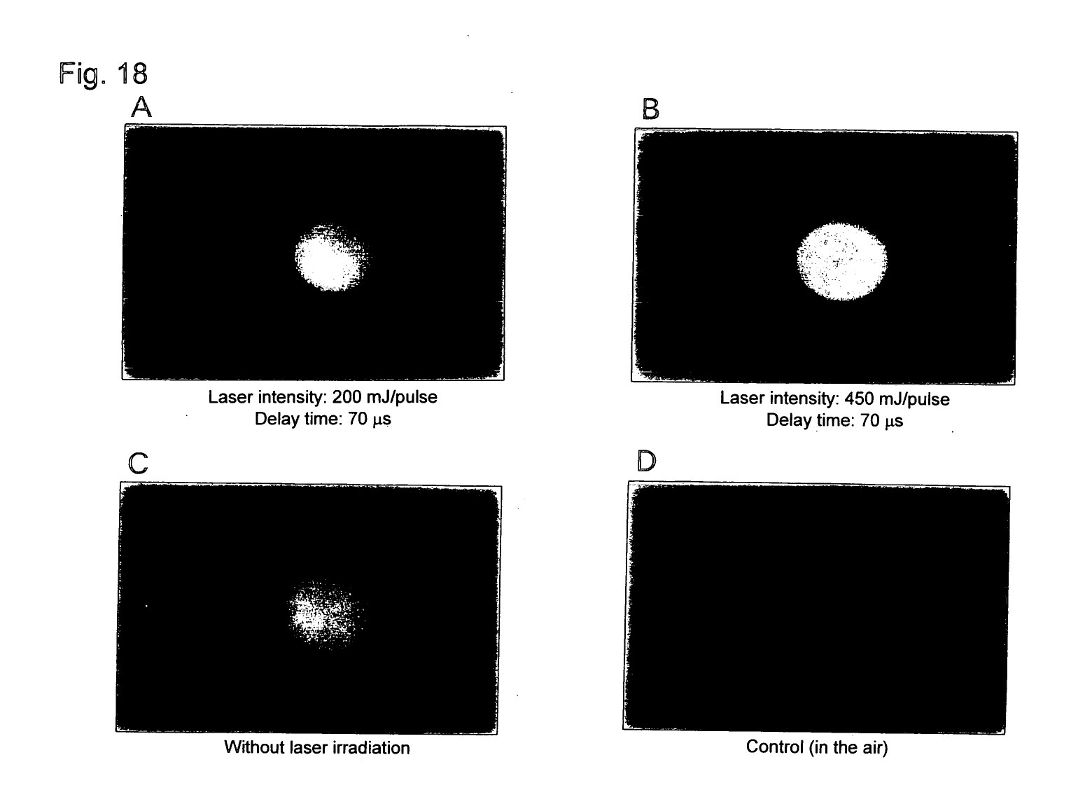

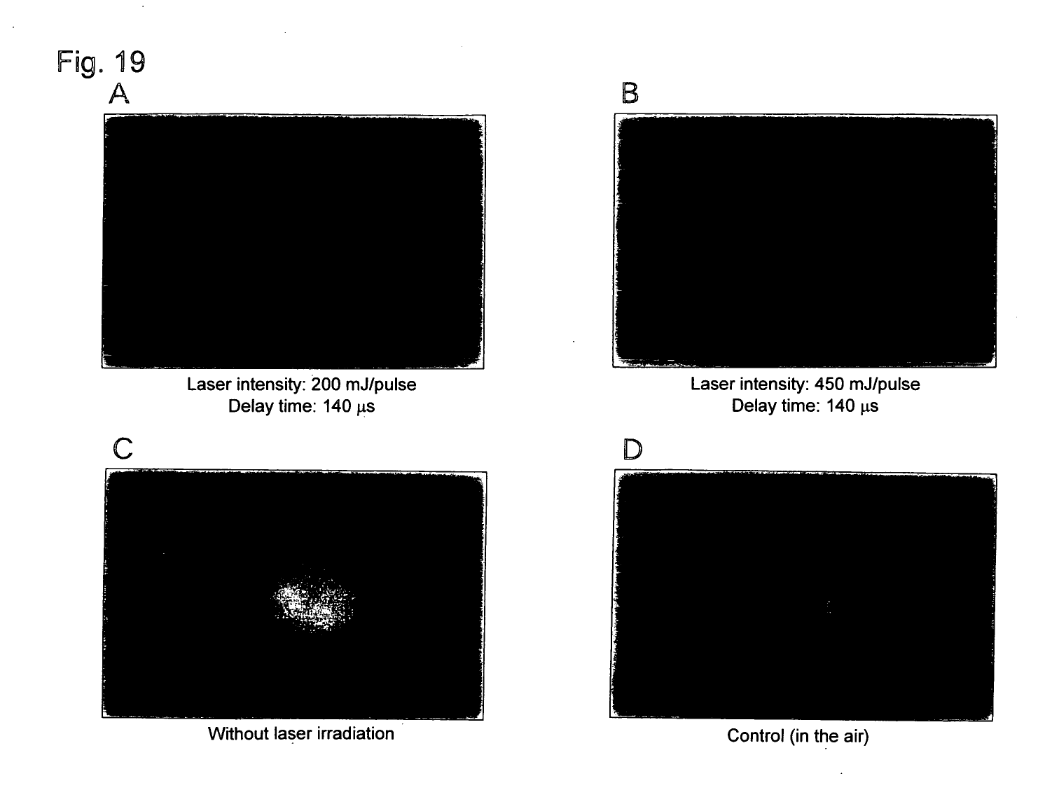

The results are shown in FIGS. 18A to 18D and 19A to 19D. FIG. 18A to 18D show the image pickup results at a delay time of 70 μs (0.05 deg) with conditions of laser intensity of 200 mJ/pulse (charging voltage 900 V), laser intensity of 450 mJ/pulse (charging voltage 1000 V), no laser irradiation (control), and in the air (control), respectively. FIG. 19A to 19D shows the image pickup results at a delay time of 140 μs (0.1 deg) with conditions of laser intensity of 200 mJ/pulse (charging voltage 900 V), laser intensity of 450 mJ/pulse (charging voltage 1000 V), no laser irradiation (control), and in the air (control), respectively. When no vapor bubbles are generated, milk exists in the vicinity of an illumination section and an observation section. Thus, illumination light emitted from the illumination section is diffused and reflected by milk and images taken at such time glow white and have high brightness. On the other hand, when small vapor bubbles are generated, images of red paper inside the silicon tube are taken so that such images look red and have low brightness. In addition, when appropriate vapor bubbles in sufficient sizes are generated, milk in the vicinity of such illumination section and such observation section is excluded. Thus, there is no more diffusion or reflection due to the presence of milk and nothing appears in images (same as a control (in the air)). That is, the condition under which nothing appears is the best condition.

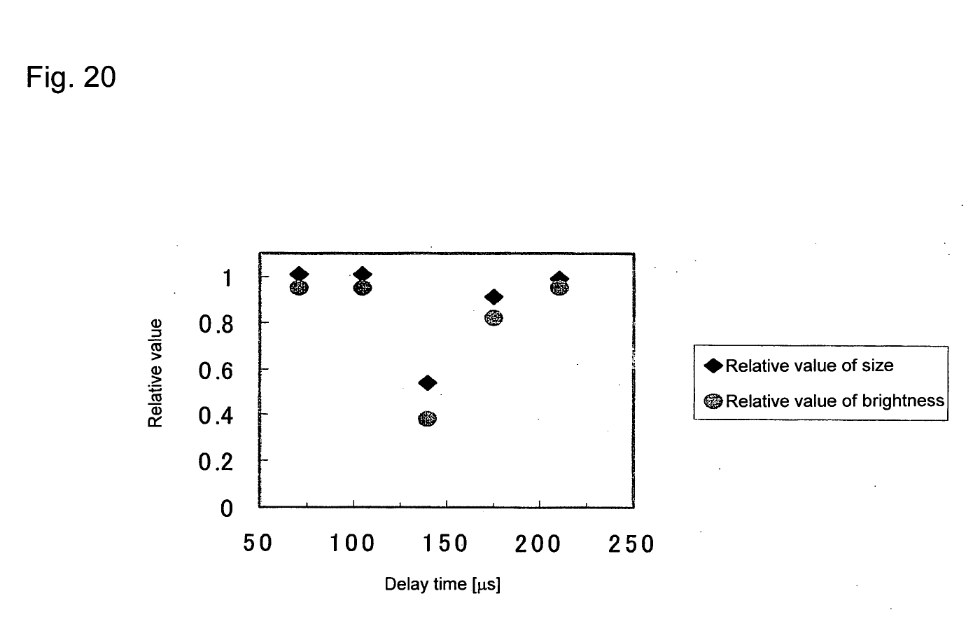

FIG. 20 shows relative values of the size and brightness at each delay time when laser intensity is 450 mJ/pulse. In addition, the fact that both the size and the brightness of an image were small indicates that vapor bubbles of sufficient sizes were generated.

In FIGS. 18A to 18D and 19A to 19D, images obtained in the control case without laser irradiation look white because no vapor bubbles were generated. When the delay time was 70 μs and when laser intensity was 200 mJ/pulse, generation of vapor bubbles was insufficient. Therefore, the image of milk looks white. When laser intensity was 450 mJ/pulse, images were taken before the obtaining of vapor bubbles with sufficient large sizes. Accordingly, the image looks red (FIG. 18). When the delay time was 140 μs, in both cases of laser intensities at 200 mJ/pulse and 450 mJ/pulse, images were taken when sufficient sizes of vapor bubbles were obtained. Therefore, nothing appears in the images as in the case of the control (in the air) (FIG. 19). In addition, when laser intensity was 450 mJ/pulse, in the cases of delay times set to 70 μs to 210 μs, both the size and the brightness of the image of the inside of the tube reached minimum levels at a delay time of 140 μs (FIG. 20). In the experiment conducted in this Example, the best visual field was obtained at a delay time of 140 μs.

### Example 6

### Observation of an Aorta Lumen of a Domestic Rabbit with the Use of an Endoscope Irradiating a Lumen with High-Intensity Pulsed Light and Generating Vapor Bubbles so as to Allow Temporary Exclusion of Liquid in the Lumen

An aorta lumen of a domestic rabbit was observed using an endoscope irradiating a lumen with high-intensity pulsed light and generating vapor bubbles so as to allow temporary exclusion of liquid in the lumen. The structure of the endoscope used was in accordance with that of the endoscope shown in FIG. 15 used in Example 4. However, a flash lamp excitation Ho:YAG laser (manufactured by Cyber Laser, model FLHY-1) was used as a laser generator. In addition, a fiber having a core diameter of 0.6 mm and an outside diameter of 1.45 mm was used as a laser irradiation fiber. The fiber was connected with an endoscope having an outside diameter of 1.3 mm (manufactured by au Medical Laboratory) for use.

A 10 Fr. sheath was placed in a domestic rabbit aorta and the above fiber connected with the endoscope was inserted therein.

The laser irradiation conditions were 10 Hz and 400 mJ/pulse. For a control case, images of the intravascular lumen were taken without laser irradiation.

When an image was taken without laser irradiation, the entire image looked red because of the presence of blood so that it was impossible to see the intravascular lumen. When the laser irradiation was performed so as to generate vapor bubbles, blood in the blood vessel in the anterior portion of the sheath was temporarily excluded by vapor bubbles and thus it was possible to observe the intravascular lumen.

It is possible to readily detect the flexed direction of the forward end of a thin tube with the use of the apparatus of the present invention by allowing the forward end of the thin tube, which is a catheter or the like that has been inserted into a lumen of a blood vessel or the like, to be subjected to light irradiation and monitoring the site at the forward end of a thin tube subjected to light irradiation or the site at which temperature increase has been observed as a result of light irradiation with the use of a sensor contained in the forward end. Further, the apparatus of the present invention comprises an actuator at the forward end of a thin tube, such actuator being deformed as a result of light irradiation. Thus, when the actuator is subjected to light irradiation so as to be deformed, it is possible to readily allow the forward end of a thin tube flexed in any direction.

In the case of the apparatus of the present invention comprising a double lumen thin tube structure, the flexed direction of the thin tube is preliminarily detected with the use of the above sensor. Next, an inner thin tube is moved in the anteroposterior direction or rotational direction in a manner such that an actuator contained in the forward end of the inner thin tube is disposed to be subjected to light irradiation, followed by light irradiation on the actuator. Thus, the actuator is deformed by light irradiation so that the forward end of the thin tube is allowed to further be flexed.

Further, in the case of the apparatus of the present invention, the forward end of a thin tube, which is a catheter or the like that has been inserted into a lumen of a blood vessel or the like, is allowed to be flexed corresponding to a state of such lumen (e.g., bending and branching) only by allowing such forward end to be subjected to light irradiation. Specifically, with the performance of light irradiation alone for a certain time, the apparatus itself detect the suitable flexed direction. Thus, it becomes possible to readily and promptly control the traveling direction of a thin tube without confirming the position of the forward end of the thin tube. In addition, it is also possible to allow such thin tube to be flexed in any direction by controlling the site of light irradiation in a lumen.

Furthermore, with the use of the thin tube of the present invention having the forward end that is allowed to be flexed, into which an endoscope apparatus comprising a high-intensity pulsed light generating means and a high-intensity pulsed light transmitting means for transmitting high-intensity pulsed light, such endoscope irradiating the inside of a lumen with high-intensity pulsed light and generating vapor bubbles so as to be able to temporarily exclude liquid in the lumen has been incorporated, it becomes possible to operate the forward end of the thin tube equipped with an observation means in a direction appropriate for observation (e.g., toward the center of the lumen) in the inside of a lumen such that the inside of the lumen can be accurately observed.

All publications, patents, and patent applications cited herein are incorporated herein by reference in their entirety.

What is claimed is:

1. A thin tube for medical use, which is inserted into a lumen of a living body so as to be used for observation or treatment of the living body, wherein when the forward end of the thin tube comes into contact with the inner wall of the lumen and is passively flexed, the forward end of the thin tube can be actively flexed by light irradiation to the side on which the forward end has been flexed.

2. The thin tube for medical use according to claim 1, wherein the lumen of a living body is a digestive tract or a blood vessel.

3. The thin tube for medical use according to claim 1, wherein the thin tube is a medical catheter.

4. The thin tube for medical use according to claim 1, wherein the thin tube is a medical endoscope.

5. The thin tube for medical use according to claim 1, comprising a device for sensing light irradiation and/or an actuator that is operated via light irradiation at the forward end of the thin tube and a light transmission means that is located in the thin tube; wherein
the forward end of the thin tube can be flexed due to the action of the device or actuator when the device and/or actuator are/is irradiated with light from the light transmission means.

6. The thin tube for medical use according to claim 5, wherein the actuator, which is operated via light irradiation and is disposed in the forward end of the thin tube, is a deformable material that can be deformed by light irradiation, and the forward end of the thin tube can be flexed as a result of deformation of the deformable material due to the action of light irradiated from the light transmission means contained in the thin tube.

7. The thin tube for medical use according to claim 6, wherein the deformable material absorbs light so as to generate heat so that the deformable material can be deformed by heat.

8. The thin tube for medical use according to claim 6, containing a light-absorbing material that absorbs light so as to generate heat and a deformable material that can be deformed by heat in a manner such that the light-absorbing material and the deformable material are allowed to come into contact with each other for thermal conduction at the forward end of the thin tube and a light transmission means; wherein
the forward end of the thin tube can be flexed by irradiating the light-absorbing material with light from the light transmission means so as to cause deformation of the deformable material due to conduction of heat that is generated from the light-absorbing material.

9. The thin tube for medical use according to claim 6, comprising a deformable material disposed in a continuous manner or at certain intervals over the whole circumference of the forward end of the thin tube.

10. The thin tube for medical use according to claim 6, wherein the deformable material that can be deformed is a bimetal or shape-memory alloy.

11. The thin tube for medical use according to claim 6, wherein the deformable material that can be deformed is a polymer gel actuator.

12. The thin tube for medical use according to claim 6, wherein the flexed angle of the forward end of the thin tube can be controlled by changing the intensity of irradiated light so as to change the strength of the deformable material to be deformed.

13. A double lumen thin tube for medical use, which is inserted into a lumen of a living body so as to be used for observation or treatment, comprising an inner thin tube and an outer thin tube, wherein the inner thin tube is the thin tube for medical use according to claim 1.

14. A method for inserting the thin tube for medical use according to any one of claims 1 to 13 into a lumen of a living body, comprising the steps of:
(a) inserting the thin tube into a lumen of a living body;
(b) irradiating a device and/or actuator of the forward end of the thin tube with light by a light transmission means that is disposed in the thin tube so as to allow the forward end of the thin tube to be flexed when the thin tube comes into contact with the inner wall of the lumen of a living body so that it becomes difficult to insert the forward end of the thin tube; and
(c) further inserting the thin tube into the lumen of a living body.
